(19) United States
(12) Patent Application Publication
(10) Pub. No.: US 2024/0091518 A1
(43) Pub. Date: Mar. 21, 2024

(54) BLOOD CONTROL VALVES, SYSTEMS, AND METHODS

(71) Applicant: Piper Access, LLC, Salt Lake City, UT (US)

(72) Inventors: Jay Allen Muse, Salt Lake City, UT (US); Ryan S. VanDyke, Layton, UT (US)

(73) Assignee: Piper Access, LLC, Salt Lake City, UT (US)

(21) Appl. No.: 18/472,208

(22) Filed: Sep. 21, 2023

Related U.S. Application Data
(60) Provisional application No. 63/408,765, filed on Sep. 21, 2022.

(51) Int. Cl. A61M 39/06; A61M 25/00; A61M 25/06

(52) U.S. Cl. CPC A61M 39/0613; A61M 25/0097; A61M 25/0606

(57) ABSTRACT

In some instances, an apparatus includes a hub that defines a cavity, a catheter that includes a lumen, and a core attached to the catheter. The core can include a valve actuation tip, and at least a portion of the core can be positioned within the cavity of the hub. The apparatus can further include a valving member that includes an attachment portion that is fixedly attached to the core and a valve positioned distal to a proximal end of the cavity and proximal to the valve actuation tip. The valve can be movable relative to the core such that as at least a portion of the valve moves distally, interaction between the valve and the valve actuation tip opens the valve to establish fluid communication between the cavity of the hub and the lumen of the catheter.

### CROSS-REFERENCE TO RELATED APPLICATIONS

This application claims priority benefit under 35 U.S.C. § 119(e) of U.S. Provisional Patent Application No. 63/408,765, filed Sep. 21, 2022, titled BLOOD CONTROL VALVES, SYSTEMS, AND METHODS, the entire contents of which are hereby incorporated by reference herein.

### TECHNICAL FIELD

Certain embodiments described herein relate generally to blood control valves, and further embodiments relate more particularly to devices, such as, for example, catheters; systems; and methods that include or pertain to blood control valves.

### BACKGROUND

Many catheters are introduced into a patient via insertion needles. Some catheter systems include a catheter that is positioned over an insertion needle prior to introduction of the catheter into the patient. At least a distal tip of the needle can extend past a distal end of the catheter, and the distal end of the catheter may be tipped so as to have a smaller diameter than does a remainder of the catheter. The distal tip of the needle can be inserted into a vessel of the patient, and the catheter can follow through the opening thus created by the needle. Some systems exist for advancing the catheter over the needle and into the vessel. Known devices, systems, and methods, however, suffer from one or more drawbacks that can be resolved, remedied, ameliorated, or avoided by certain embodiments described herein.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a perspective view of an embodiment of a catheter delivery system in a pre-use, retracted, undeployed, or pre-actuation operational state;

FIG. 2 is a cross-sectional view of the catheter delivery system of FIG. 1, taken along the view line 2-2 in FIG. 1, in the pre-use, retracted, undeployed, or pre-actuation operational state;

FIG. 3 is a perspective view of a lower end of an embodiment of an upper housing element compatible with the catheter delivery system;

FIG. 4 is an enlarged view of the upper housing element of FIG. 3 taken along the view line 4 in FIG. 3;

FIG. 5 is a perspective view of an embodiment of a lower housing element compatible with the catheter delivery system;

FIG. 6A is a perspective view of an embodiment of a catheter connection hub compatible with the catheter delivery system;

FIG. 6B is a cross-sectional view of the catheter connection hub of FIG. 6A taken along the view line 6B-6B in FIG. 6A;

FIG. 7A is a perspective view of an embodiment of a catheter subassembly compatible with the catheter delivery system;

FIG. 7B is a cross-sectional view of a proximal portion of the catheter subassembly of FIG. 7A taken along the view line 7B-7B in FIG. 7A;

FIG. 8A is a perspective view of an embodiment of a valving member that is compatible with embodiments of the catheter subassembly of FIG. 7A;

FIG. 8B is another perspective view of the valving member of FIG. 8A;

FIG. 8C is a cross-sectional view of the valving member of FIG. 8A taken along the view line 8C-8C in FIG. 8A;

FIG. 8D is an end-on plan view of a proximal end of the valving member of FIG. 8A;

FIG. 9A is a perspective view of an embodiment of a valve actuation member that is compatible with the catheter of subassembly of FIG. 7A;

FIG. 9B is a cross-sectional view of the valve actuation member of FIG. 9A taken along the view line 9B-9B in FIG. 9A;

FIG. 10A is a perspective view of an embodiment of a strain-relief member that is compatible with the catheter subassembly of FIG. 7A;

FIG. 10B is a cross-sectional view of the strain-relief member of FIG. 10A taken along the view line 10B-10B in FIG. 10A;

FIG. 11 is a perspective view of an embodiment of an upper actuator compatible with the catheter delivery system;

FIG. 12 is a perspective view of an embodiment of a lower actuator or stiffener hub compatible with the catheter delivery system;

FIG. 13 is a cross-sectional view of the lower actuator of FIG. 12 and an embodiment of a stiffener in a fixedly secured state, which may be referred to collectively as a stiffener assembly;

FIG. 14 is a cross-sectional view of a distal portion of the catheter delivery system of FIG. 1 in the pre-use, retracted, undeployed, or pre-actuation state, which state is also depicted in FIGS. 1 and 2;

FIG. 15A is an enlarged cross-sectional view of an intermediate region of the catheter delivery system in the initial operational state depicted in FIG. 14, taken along the view line 15A in FIG. 14

FIG. 15B is an enlarged cross-sectional view of a distal end of the catheter delivery system in the initial operational state depicted in FIG. 14, taken along the view line 15B in FIG. 14;

FIG. 16 is another cross-sectional view of the catheter delivery system of FIG. 1, similar to the view shown in FIG. 2 but depicting the catheter delivery system in a partially deployed or preliminarily actuated state;

FIG. 17 is another cross-sectional view of the catheter delivery system of FIG. 1, similar to the view shown in FIG. 2 but depicting the catheter delivery system in a nearly fully deployed or almost fully actuated state just prior to an embodiment of a catheter assembly transitions to a coupled orientation;

FIG. 18 is another cross-sectional view of the catheter delivery system of FIG. 1, similar to the view shown in FIG. 2 but depicting the catheter delivery system in a fully deployed or fully actuated state in which the catheter assembly is in a coupled orientation, wherein the catheter delivery system includes a non-return feature that prevents retraction of a stiffener relative to a housing after full deployment is achieved;

FIG. 19 is another cross-sectional view of the catheter delivery system of FIG. 1, similar to the view shown in FIG. 2 but depicting the catheter delivery system in the fully deployed or fully actuated state and with the upper actuator having been retracted;

FIG. 20 is another cross-sectional view of the catheter delivery system of FIG. 1, similar to the view shown in FIG. 2 but depicting the catheter delivery system in the fully deployed or fully actuated state, with the upper actuator having been retracted, and with the catheter assembly having been decoupled from a handle or housing portion of the catheter delivery system; stated otherwise, FIG. 20 is a cross-sectional view of an insertion assembly portion of the catheter delivery system in a fully actuated state with the catheter assembly portion of the catheter delivery system having been removed therefrom;

FIG. 21 is a cross-sectional view of the catheter assembly in the coupled or assembled state after such has been decoupled from the insertion assembly portion of the catheter delivery system (e.g., shown in FIG. 20);

FIG. 22A is a cross-sectional view of the assembled catheter system of FIG. 21, which includes the hub of FIG. 6A connected to a catheter assembly of FIG. 7A, being connected with a fluid transfer device (specifically, a syringe), of which only a distal portion is shown;

FIG. 22B is a further cross-sectional view of the assembled catheter system of FIG. 21 after the fluid transfer device has been advanced distally relative to the catheter assembly sufficiently far to open the valving member of the catheter assembly;

FIG. 23 is a cross-sectional view of another embodiment of an assembled hub and catheter assembly;

FIG. 24A is a perspective view of an embodiment of a valving member that is compatible with the catheter assembly of FIG. 23;

FIG. 24B is an end-on plan view of a proximal end of the valving member of FIG. 24A;

FIG. 25 is a perspective view of an embodiment of a core that is compatible with the catheter assembly of FIG. 23;

FIG. 26 is a cross-sectional view of another embodiment of a catheter assembly that includes a hub integrally formed with a valve actuation member; and

FIG. 27 is a cross-sectional view of another embodiment of a catheter assembly that includes a hub integrally formed with a valve actuation member.

### DETAILED DESCRIPTION

The present disclosure relates generally to blood control devices, systems, and methods. Certain embodiments of blood control devices are disclosed herein in the context of catheter systems, including systems for delivering catheters into the vasculature of patients. While certain blood control devices, systems, and methods described herein are well suited for use in such catheter and catheter delivery systems, they are not necessarily limited to these contexts are can be advantageous in a variety of other suitable environments.

Illustrative examples of catheter delivery systems into which blood control valves of the present disclosure can be used advantageously include over-the-needle catheter placement systems. For example, certain embodiments of over-the-needle catheter systems into which blood control valves of the present disclosure may be suitably incorporated are described in U.S. Pat. No. 11,065,419, titled CATHETER DELIVERY DEVICES, SYSTEMS, AND METHODS, which issued on Jul. 20, 2021 (hereafter U.S. Pat. No. 11,065,419), the entire contents of which are hereby incorporated by reference herein.

As discussed in U.S. Pat. No. 11,065,419, in some embodiments, a catheter delivery system can include a multi-part hub that is assembled during delivery of a catheter into the vasculature of a patient. In particular, a first hub component, which may also be referred to as a connection hub, can be removably secured to a handle, while a second hub component (or set of hub components), which may also be referred to as a core, is permanently affixed to a catheter. The core and catheter can be movable relative to the handle during placement of the catheter within the vasculature of a patient. An actuator that is movable relative to the handle may be coupled with the core so as to advance the core and catheter relative to the connection hub. The core may be advanced into connection with the connection hub. For example, the core may be advanced into the connection hub until a secure connection is established, such as via a plurality of arms resiliently snapping into place. The hub may subsequently be removed from the handle (e.g., the handle may be rotated relative to the connection hub to disengage a threaded interface). As a result, an assembled catheter assembly, which includes the catheter, the core, and the hub, can remain in place in the patient, with the catheter extending into the patient vasculature and the hub and core remaining external to the patient. Access to the patient vasculature can be achieved by coupling a fluid exchange device of any suitable variety to the catheter hub. For example, in some instances, a syringe or other suitable device can be coupled to the hub for delivery of fluid (e.g., medication) to or removal of fluid (e.g., blood) from the vasculature.

As disclosed in U.S. Pat. No. 11,065,419, certain catheter delivery systems can include a catheter, such as just described, and a needle that extends through a distal end of the catheter. In many cases, the catheter is attached to at least a distal end of the needle in a pre-use state. In certain embodiments, a catheter delivery system can include a stiffener that can assist in advancement of the catheter relative to the needle and can remain extended relative to the needle when the stiffener and the needle are removed from the catheter. The stiffener can thereby act as a shield to prevent inadvertent contact with the distal tip of the needle. That is, the stiffener can function as a safety shield to prevent needle sticks. In some embodiments, the stiffener may be locked in an advanced position to ensure such shielding.

In some instances, it may be desirable for catheter systems such as described above to include a blood control valve. The blood control valve can prevent blood from egressing through the catheter and out of the hub when the hub is in an uncoupled state relative to a fluid exchange device, a cap, or other suitable device that, when coupled with the hub, effectively seals the hub. For example, the blood control valve can prevent blood from egressing from a placed catheter during a period following insertion of a catheter assembly into the vasculature of a patient and that extends until a fluid control device, such as a syringe, is secured in a fluid-sealed state to a hub of the catheter assembly. Certain embodiments described herein are particularly suitable for use with multi-component catheter hubs that are assembled together during delivery of the catheter into the blood vessel, as previously described. Some embodiments are particularly well suited for power injection. That is, the catheters, hubs, and blood valve components are functional at power injection pressures, or stated otherwise, can be used for power injections without subsequent loss of functionality. The blood control valves, for example, can maintain a seal after a power injection event.

Although much of the present disclosure is made in the context of catheter systems that include multi-part hubs that are assembled during placement of the catheter, in other embodiments, the hubs may be preassembled. For example, some embodiments include pre-assembled hubs that include blood control valves. In some embodiments, the hubs may be formed of a unitary monolithic component that is attached to the catheter.

Moreover, known blood control valves can suffer from a variety of drawbacks. For example, some blood control valves have dead space configurations (size, shape, etc.) that may be prone to bacterial growth.

Certain embodiments described herein can be particularly well suited to ameliorate or eliminate one or more drawbacks of known catheters, catheter placement systems, and/or blood control valves. Some embodiments permit multi-component hubs that are assembled during placement of a catheter by a practitioner to include blood control capabilities. Various embodiments provide one or more of the foregoing and/or one or more additional and/or other advantages, as will be apparent from the present disclosure

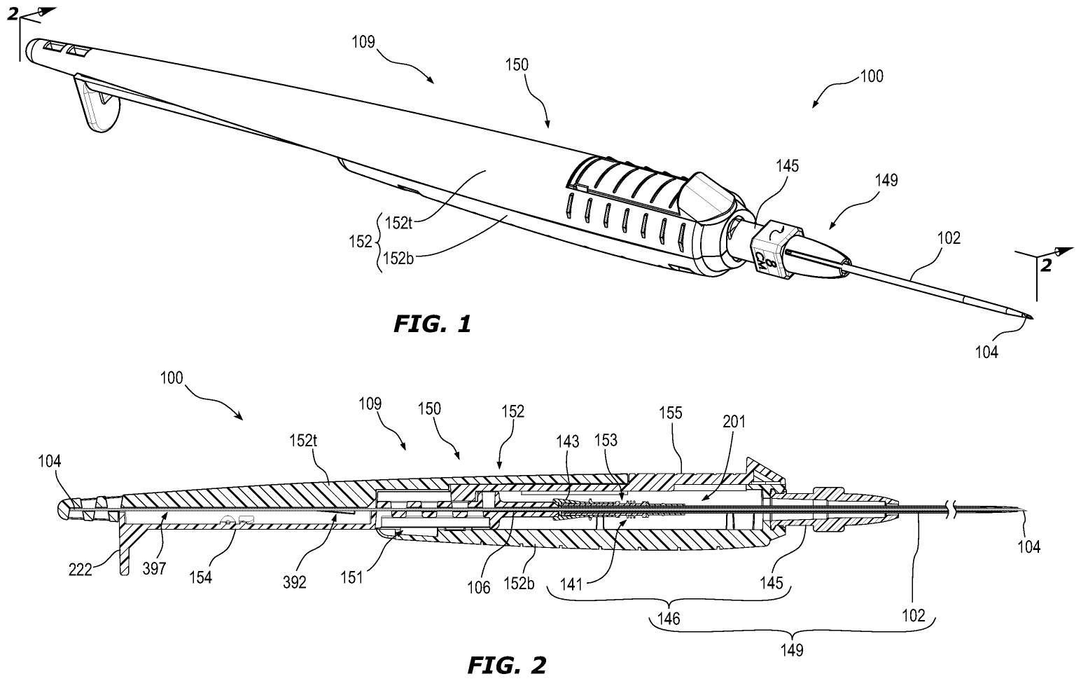

FIGS. 1 and 2 depict an embodiment of a catheter delivery system 100. In some embodiments, certain components of the catheter delivery system 100 can resemble clearly analogous components of various embodiments described in U.S. Pat. No. 11,065,419. Accordingly, like features may be designated with like reference numerals, with the leading digits altered, where appropriate. Relevant disclosures set forth in U.S. Pat. No. 11,065,419 regarding similar and/or similarly identified features are hereby incorporated by reference herein with respect to the system 100.

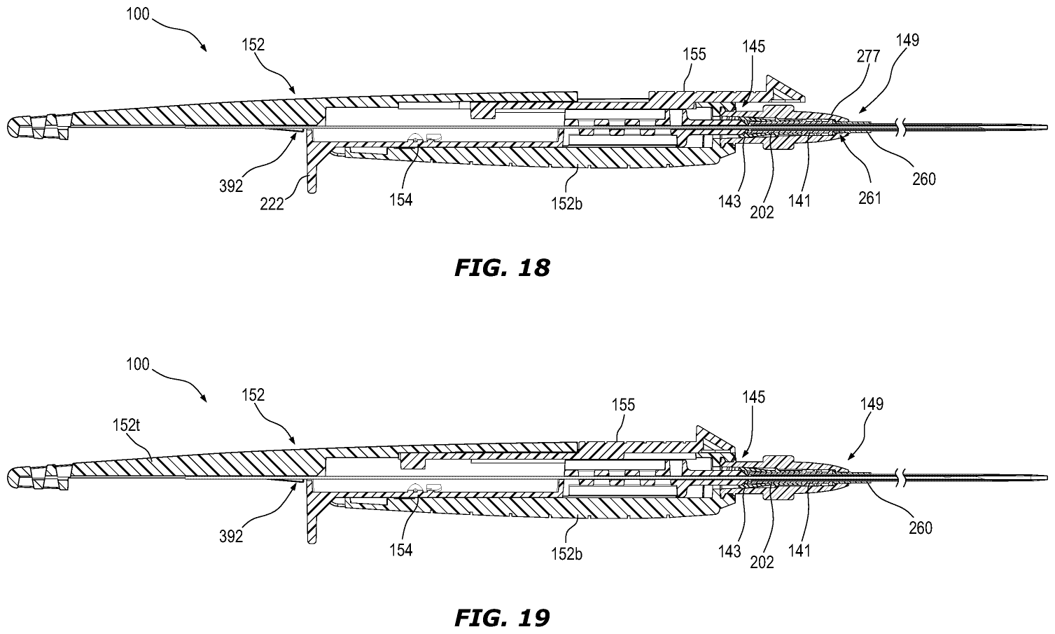

In the illustrated embodiment, the system 100 includes an insertion assembly 109 that is selectively coupled with a catheter assembly 149. As further discussed below, the system 100 is in a pre-use, retracted, undeployed, or pre-actuation operational state. The insertion assembly 109 is configured to deploy a catheter 102 to a desired depth within a vessel of a patient by advancing the catheter 102 over an insertion needle 104. In so doing, the insertion assembly 109 transitions the catheter assembly 149 from a disassembled state to an assembled state, as further discussed below. After deployment of the catheter 102 and after transition of the catheter assembly 149 to its assembled state, the insertion assembly 109 can be detached from the fully assembled catheter assembly 149 and withdrawn therefrom, thus leaving the catheter assembly 149 in place, with the catheter positioned within the vasculature of the patient. Deployment of the catheter 102 and transition of the catheter assembly 149 to the assembled state are events of which at least a portion may occur simultaneously. For example, assembly of a hub portion of the catheter assembly 149 may occur during a final phase of deployment of the catheter 102, as discussed further below (e.g., with respect to FIGS. 17 and 18).

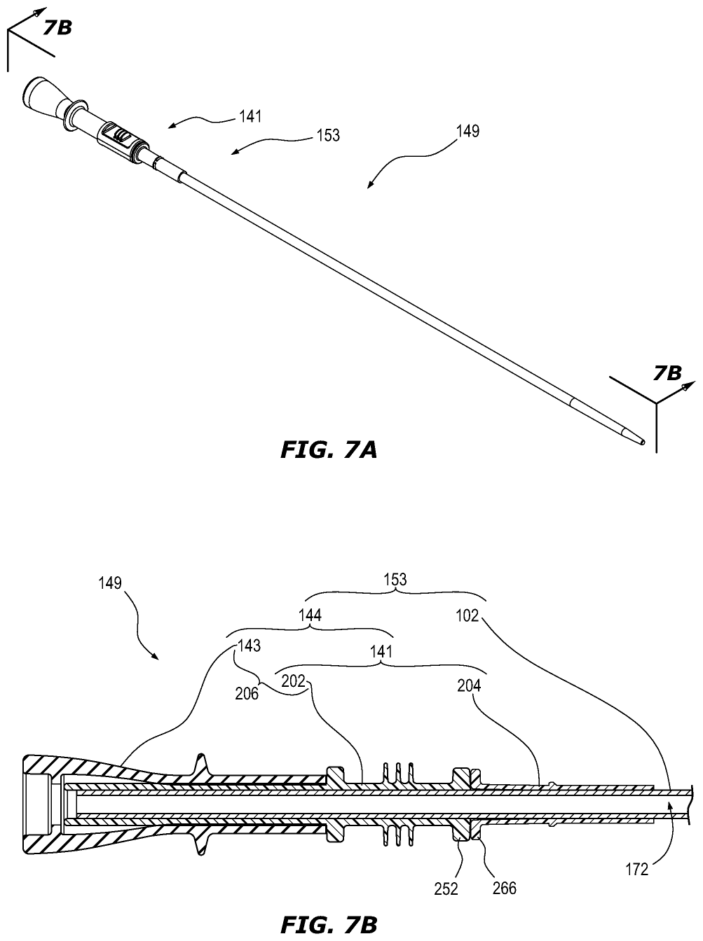

The catheter assembly 149 can include the catheter 102, a catheter hub core 141, a valving member 143, and a catheter connection hub 145. The valving member 143 may also be referred to as a valve or as a seal member. The catheter hub core 141, which may include one or more components (as discussed below), is secured to the catheter 102 in any suitable manner (e.g., overmolded over a proximal region of the catheter 102), and the valving member 143 can be coupled with the catheter hub core 141 in any suitable manner, such as discussed further below, e.g., with respect to FIG. 7B. In the illustrated disassembled state of the catheter assembly 149, which can correspond to the undeployed state of the system 100 (shown in FIGS. 1 and 2), the catheter hub core 141 is spaced from and positioned rearward of (proximal to) the catheter connection hub 145, and the catheter 102 extends through an entirety of the catheter connection hub 145. The catheter hub core 141, the valving member 143, and the catheter connection hub 145 can be assembled together to form a catheter hub 146, as shown in FIG. 21. Stated otherwise, FIG. 21 depicts the catheter assembly 149 in a fully assembled state. In this state, an assembled catheter hub 146 includes the catheter connection hub 145 fixedly secured to the catheter hub core 141. The valving member 143 forms a fluid-tight seal with the catheter connection hub 145 and further permits selective opening and closing a fluid path that extends through a lumen 172 of the catheter 102, as discussed further below.

With reference again to FIGS. 1 and 2, in the illustrated embodiment, the insertion assembly 109 includes a handle 150, which can comprise a housing 152. The housing 152 can be shaped to have an ergonomic contour that can be readily gripped by a hand of a user. The illustrated housing 152 includes a top or upper housing element 152t and a bottom or lower housing element 152b. The housing elements 152t, 152b can cooperate to define an opening, port, or channel 151 through which a stiffener hub 154 extends. The channel 151 is positioned at a mid or intermediate region of the housing 152.

In the pre-use, pre-deployment, initial, or as-packaged state depicted in FIGS. 1 and 2, the housing 152 is connected at a distal end thereof with the catheter connection hub 145. The catheter connection hub 145 can be selectively releasable from the housing 152. For example, in the illustrated embodiment, a distal end of the housing 152 can include internal threading 211, and a proximal end of the catheter connection hub 145 can include external threading 175 that is complementary to and selectively attachable to the threading 211 of the housing 152 via rotational engagement. (See, e.g., FIGS. 2, 3, 5, 6A and 6B.) Any other suitable connection interface is contemplated.

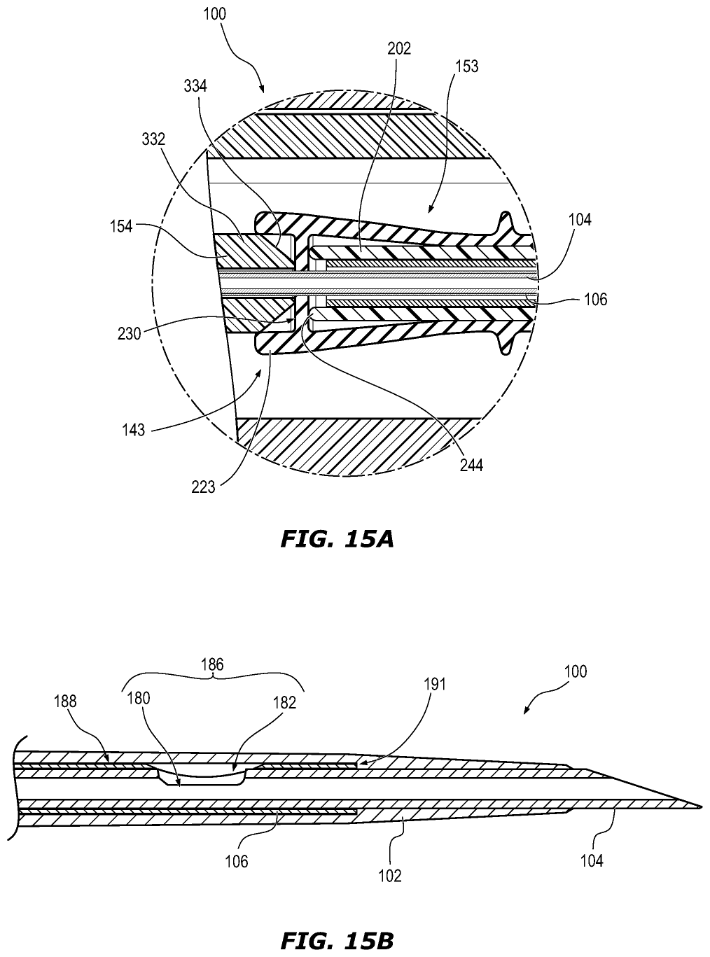

The insertion assembly 109 can further include a stiffener 106, which may also or alternatively be referred to as, or may have an alternate form that comprises at least one component that may be referred to as, a support, column, reinforcement, frame, scaffold, prop, strut, brace, spine, rod, tube, and/or cannula. For example, in the illustrated embodiment, the stiffener 106 may also be referred to as a sheathing cannula, a cannular stiffener, etc. In the illustrated embodiment, the stiffener 106 is formed of an elongated tube that is positioned between an outer surface of the insertion needle 104 and an inner surface of the catheter 102 when the system 100 is in the undeployed configuration (such as depicted in FIGS. 1 and 2). This nested or telescoped arrangement in the undeployed state is also depicted in FIG. 15B.

The stiffener 106 may be flexible in the transverse dimension (e.g., in directions orthogonal to a longitudinal axis of the tube), yet may be substantially rigid or stiff in the axial direction to counteract axial forces (i.e., longitudinally directed force) applied thereto by the distal portion of the catheter 102 during insertion of the system 100 through the vessel wall and during advancement of the system 100 through the lumen of the vessel.

With reference to FIG. 2, the insertion needle 104 is positioned within the stiffener 106 and extends through an entirety of the catheter 102 (e.g., extends distally past a forward end of the catheter 102 and extends proximally past a rearward end of the catheter 102), through an entirety of the stiffener 106, and through most of the housing 152. In particular, the needle 104 extends through a distal end of the housing 152 and a proximal end of the needle 104 is attached, internally, to a proximal end of the upper housing element 152t.

With reference to FIGS. 2 and 13, the illustrated stiffener 106 is secured at a proximal end thereof to the stiffener hub 154, which is movable within and relative to the housing 152. In the illustrated embodiment, the stiffener 106 is formed as a tubular element that encompasses a portion of the needle 104. The stiffener 106 is movable relative to (e.g., over, along an exterior of) the needle 104.

The stiffener hub 154 can include an actuation element 222 that may protrude away from the housing 152 to be engageable by the hand (e.g., one or more fingers) of a user. For example, the user may press distally on the actuation element 222 to thereby move the stiffener hub 154 and the attached stiffener 106 in a distal direction relative to the needle 104 and housing 152. The stiffener hub 154 may generally be referred to as an actuator, such as in the illustrated embodiment in which the stiffener hub 154 is rigidly fixed to (e.g., integrally formed with) the actuation element 222 such that the stiffener hub 154 and the actuation element 222 move in unison as a single body. Alternatively, the stiffener hub 154 may be said to be coupled to (e.g., integrally formed with or otherwise) an actuator. Thus, in the illustrated embodiment, a body portion of the stiffener hub 154 may be said to be attached to the actuation element 222. The actuation element 222 may also or alternatively be referred to as an actuator, a deployment actuator, an advancement actuator, a primary actuator, a first actuator, a direct stiffener hub actuator, a lower actuator, a rear actuator, etc. Moreover, in many instances, reference to the actuator 222 may more generally be understood as a reference to the stiffener hub 154 in its entirety. Further still, the stiffener hub 154 may be referred to as a slider, as it may, in some instances, slide within the housing 152.

With continued reference to FIG. 2, the insertion assembly 109 can further include an initiation actuator 155, which is described further below. The initiation actuator 155 may also be referred to as an insertion actuator, a stabilization actuator, a supplemental actuator, an optional actuator, a second actuator, an indirect stiffener hub actuator, an upper actuator, a forward actuator, a slider, etc. With respect to the designation of the actuators 222, 155 as lower or upper actuators, respectively, it should be understood that these terms refer to the positions depicted in the views shown in FIGS. 1 and 2, and are not limiting with respect to other arrangements. That is, the terms “upper” and “lower” are used illustratively herein for convenience, it being understood that these terms are readily substituted with other suitable appellations for the actuators 222, 155. For example, in other embodiments, the actuators 222, 155 are reversed, such that the actuator 222 is accessible at an upper end of the housing 152 and the actuator 155 is accessible at the lower end of the housing 152. In still other embodiments, the positions of the actuators 222, 155 may be fully altered, such as by being at lateral positions. For example, rather than being at opposing or opposite upper and lower sides of the housing 152, the actuators 222, 155 can be positioned at other opposing sides (e.g., left and right sides) of the housing 152.

The initiation actuator 155 can selectively couple with the stiffener hub 154 to move the stiffener hub 154 forward by an initial amount, as further discussed below. In the illustrated embodiment, when the system 100 is in the pre-use or pre-deployment configuration, the initiation actuator 155 can be adjacent to or in coupling contact with the stiffener hub 154 (see FIG. 2) such that forward or distal movement of the actuator 155 effects immediate (or nearly immediate, such as where the initiation actuator 155 moves a short distance prior to engaging the stiffener hub 154), simultaneous or concurrent forward movement of the stiffener hub 154 (and, thereby, forward movement of the stiffener 106 and catheter 102).

In some instances, the system 100 can be fully deployed using only the lower actuator 222. In other instances, the system 100 can be deployed in two separate phases: first, by advancing the upper actuator 155 to insert the catheter 102 to a first depth within the vessel of a patient; and second, by advancing the lower actuator 222 to further advance the catheter 102 to a second depth within the vessel that is greater than the first depth. Embodiments of the latter deployment technique are discussed below with respect to FIGS. 2 and 16-21.

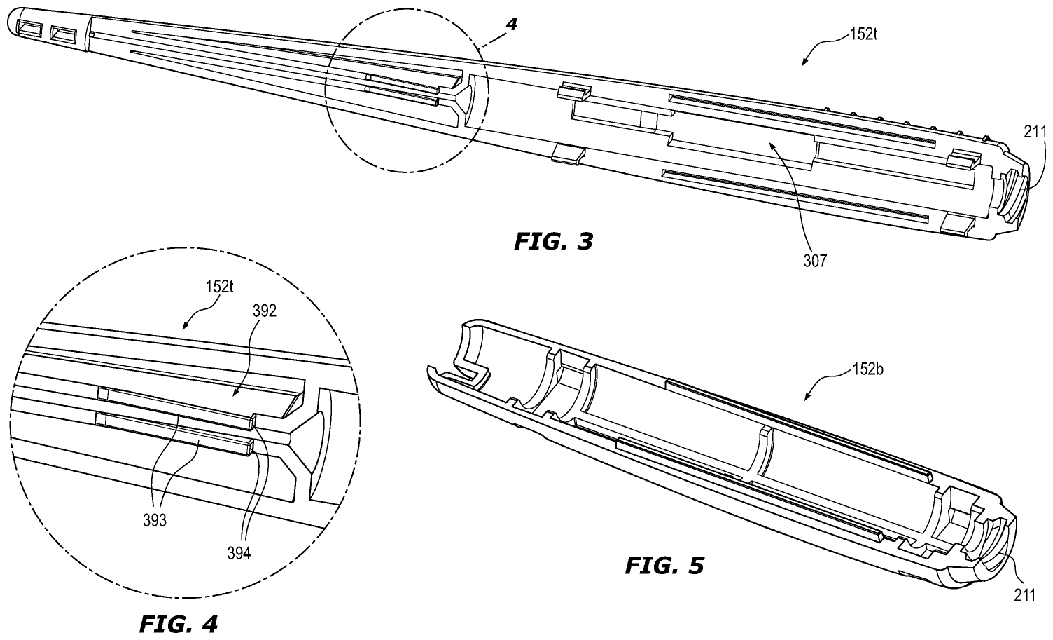

With reference to FIGS. 3-5, in some embodiments, one or more of the upper and lower housing elements 152t, 152b, the upper actuator 155 and/or the stiffener hub 154 are formed of a polycarbonate (PC) and acrylonitrile butadiene styrene (ABS) blend (i.e., PC/ABS). In other embodiments, one or more of the aforementioned components are formed of polycarbonate, acetal, etc. Any other suitable material and/or combinations thereof is/are contemplated.

The upper and lower housing elements 152t, 152b can be joined together in any suitable fashion. The upper actuator 155 and the stiffener hub 154 can be coupled with the housing 152 in any suitable manner so as to be moveable (e.g., translatable) relative thereto.

With reference to FIG. 4, the upper housing element 152t can define a stop 392, which can inhibit or prevent proximal movement of the stiffener hub 154, and thus proximal movement of the attached stiffener 106, after the stiffener hub 154 and stiffener 106 have been distally advanced by a sufficient amount relative to the needle 104, as discussed further below. The illustrated stop 392 includes a pair of ramps 393, each of which includes an abutment or engagement surface 394 at a distal end thereof. The ramps 393 of the stop 392 can project downwardly.

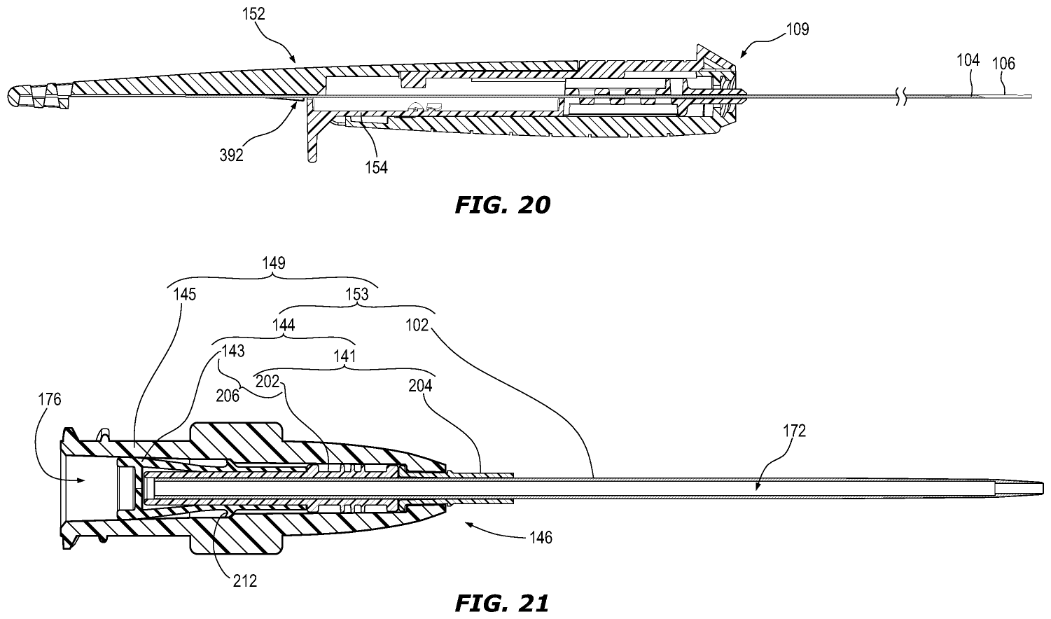

The stop 392 may be referred to as a non-return, locking, shielding, or safety feature. That is, the non-return feature prevents retraction of the stiffener 106 relative to the housing 152 after full deployment of the system 100. As further discussed below, once a proximal end of the stiffener hub 154 is advanced distally past the engagement surfaces 394 of the ramps 393, the engagement surfaces 394 interface with a proximal surface of the stiffener hub 154 to prevent the stiffener hub 154 from moving proximally relative to the housing 152. The stiffener hub 154 thus cannot be retracted relative to the housing 152. Thus, the stiffener 106 to which the stiffener hub 154 is attached is maintained in a fixed, shielding position over the needle tip (e.g., in a shielding position such as that depicted in FIG. 20).

In the illustrated embodiment, the stiffener 106 is maintained in the fully deployed position, and thus extends a maximum length past the needle tip. In other embodiments, the non-return feature may permit some amount of proximal movement of the stiffener hub and stiffener relative to the housing after deployment, but prevent full retraction of the stiffener from over the needle. That is, the system can maintain at least some length of the stiffener past the distal end of the needle in an amount sufficient to shield the needle tip from inadvertent contact. Stated otherwise, the system can retain the stiffener in at least a partially deployed state.

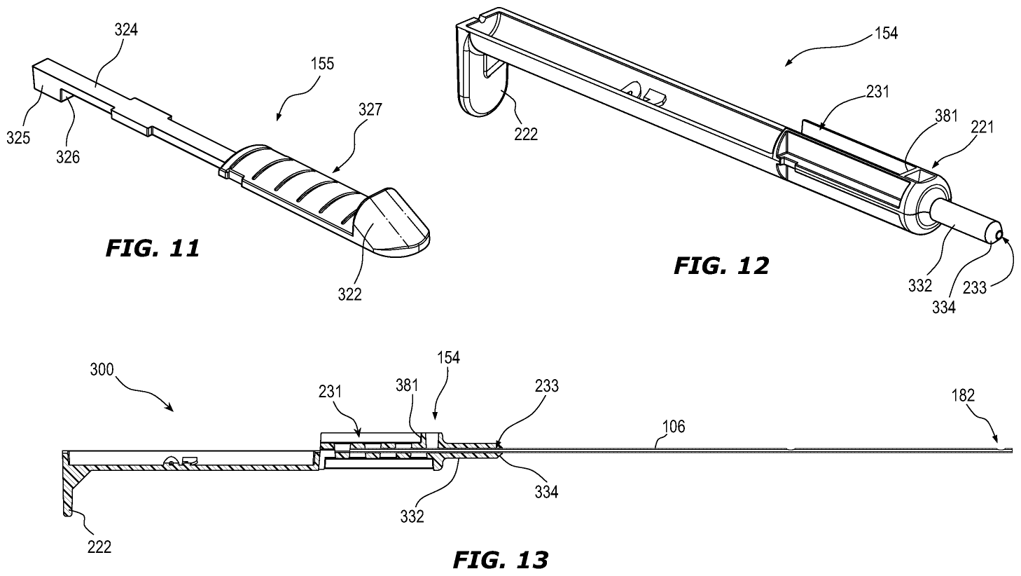

With reference to FIG. 11, the upper actuator 155 can include an actuation region 327 engageable by a user to advance the upper actuator 155 distally. In further embodiments, the actuation region 327 may include friction enhancing features, which may facilitate distal and/or proximal movement of the upper actuator 155. In some embodiments, a catch 322 can extend upwardly at a distal end of the actuation region 327.

The upper actuator 155 can include a longitudinal stem 324 that connects the actuation region 327 with an engagement protrusion 325. The stem 324 may also be referred to as a guide or slider. The engagement protrusion 325 that extends from the stem 324 can be configured to engage the stiffener hub 154 within the housing 152, as discussed further below. For example, in the illustrated embodiment, the engagement protrusion 325 includes an engagement face 326 that interferes with a surface of the stiffener hub 154 when the upper actuator 155 is advanced distally. The illustrated engagement face 326 is a substantially planar face at a distal end of the engagement protrusion 325 which, when positioned within the assembled system 100, extends substantially transverse or orthogonal to the longitudinal axis of the system 100.

The longitudinal stem 324 can be sized (e.g., can define a width) to fit within a track 307 of the upper housing element 152t (FIG. 3). As the upper actuator 155 is advanced or retracted along the track 307, the longitudinal stem 324 can slide or otherwise translate within the track 307. The track 307 can constrain movement of the upper actuator 155, and in particular, can constrain movement of the longitudinal stem 324. For example, the track 307 can inhibit or prevent lateral movement of the longitudinal stem 324.

With reference to FIGS. 12 and 13, the stiffener hub 154 includes a body 221 that can define an internal channel 233 for coupling with the stiffener 106. The body 221 can define a track, guide path, or channel 231 at an upper end thereof through which the engagement protrusion 325 of the upper actuator 155 (FIG. 11) can pass. The engagement protrusion 325 can be sized to translate within the channel 231 in an uninhibited manner. The engagement face 326 of the engagement protrusion 325 (FIG. 11) can interface with (e.g., interfere with) an engagement face 381 at a distal end of the channel 231 to effect forward movement of the stiffener hub 154. For example, in the illustrated embodiment, the engagement face 381 defines a substantially planar surface that extends substantially orthogonally relative to a longitudinal axis of the assembled system 100. The opposing engagement faces 326, 381 of the channel 321 of the stiffener hub 154 and of the engagement protrusion 325 of the upper actuator 155, respectively, thus can interfere with each other, or selectively engage with each other, when the upper actuator 155 is advanced distally. The engagement faces 326, 381 can disengage from each other when the upper actuator 155 is retracted proximally.

The stiffener hub 154 can further include a protrusion 332 that extends distally from the body 221. The protrusion 332 can include a tapered distal tip 334. In the illustrated embodiment, the distal tip 334 is substantially shaped as a cone or, in some instances, a frustocone with a small rounded or planar ring at the distalmost end thereof. The tapered tip 334 of the stiffener hub 154 can directly engage the valving member 143, as further discussed below with respect to FIG. 15A, and may function as (and be referred to as) an engagement member.

The body 221 of the stiffener hub 154 can be configured to readily pass through an internal chamber 201 defined the housing 152 (see FIG. 2). The chamber 201 may be said to constrain movement of the body 221 therein, such as to ensure a substantially linear path of movement that is substantially aligned with the longitudinal axis of the system 100. As previously noted, in some instances the body 221 can be sized to slide with in the chamber 201, and the stiffener hub 154 may be referred to as a slider.

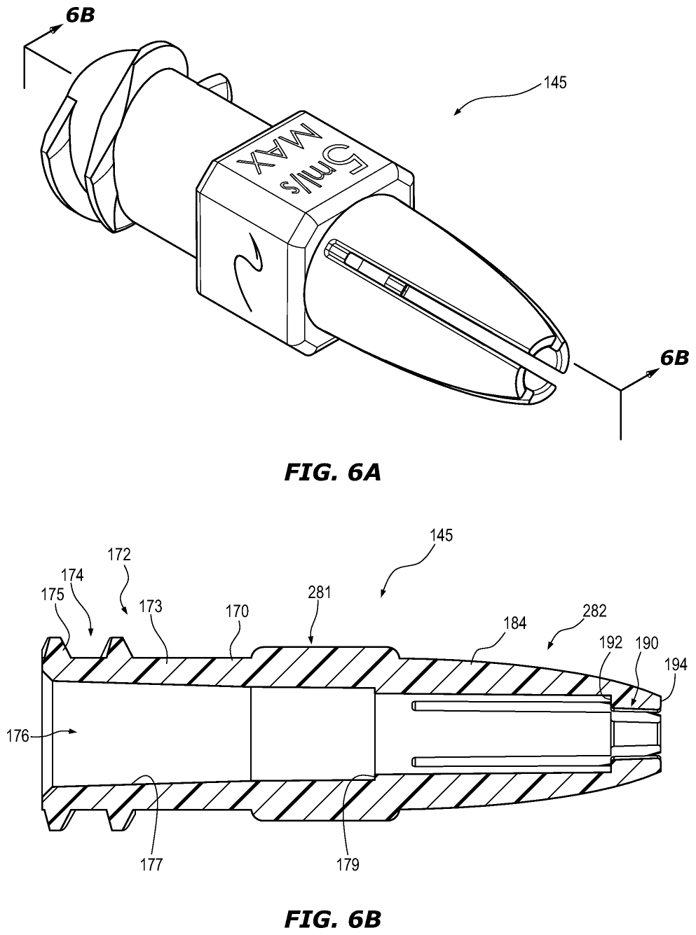

With reference to FIGS. 6A and 6B, the illustrated embodiment of a connection hub 145 includes a body 170 that defines a medical interface or medical connector 172 via which the catheter connection hub 145 can be coupled with any suitable medical device, such as, for example, a syringe or other fluid transfer device. In the illustrated embodiment, the medical connector 172 is formed as a female luer fitting 173, which can couple, for example, with any medical fluid component that includes a complementary male luer fitting. In particular, the medical connector 172 can include any suitable connection interface 174, which comprises external threading 175 in the illustrated embodiment. Moreover, the body 170 can define a cavity or lumen 176 through which fluid may pass. In the illustrated embodiment, the portion of the lumen 176 associated with the connection interface 174 defines a luer taper 177. As discussed further below, the lumen or cavity 176 can receive at least a portion of the core 141 and/or the valving member 143 when the catheter assembly 149 is fully assembled, as depicted in FIG. 21.

The body 170 can further define a base 281 at a distal end of the medical connector 172. In some embodiments, the base 281 comprises the distalmost portion of the medical connector 172. In the illustrated embodiment, the base 281 is a region of the body 170 that extends distally from the medical connector 172. The body 170 can define a hub connection interface 282 that is configured to interact with a connection interface of the core 141, as further discussed below, to secure the catheter connection hub 145 to the catheter hub core 141. In the illustrated embodiment, the connection interface 282 includes a plurality of resiliently flexible arms 184 that extend distally from the base 281. The illustrated embodiment includes four resiliently flexible arms 184. Other embodiments include more or fewer arms. Each resiliently flexible arm 184 includes an inward protrusion or catch 190 that is configured to directly interact with the connection interface 146 to secure the catheter connection hub 145 to the catheter hub core 141. In particular, the catches 190 can automatically and resiliently spring inwardly into a connection region 280, such as a region bounded by a stop and a rim, defined by the catheter hub core 141 to secure the catheter connection hub 145 to the catheter hub core 141, as further discussed below. The catches 190 can each include a proximal face 192 and a distal face 194 that can interact with the proximal and distal sidewalls of the connection region 280 of the core 141 to prevent distal or proximal movement, respectively, of the core 141 relative to the connection hub 145 once the catches 190 have been positioned between the stop and rim of the core 141 (e.g., which may be defined, in some embodiments, by a strain-relief member 204), as depicted in FIG. 21.

Other connection interfaces via which the catheter hub core 141 and the catheter connection hub 145 can be joined together are also contemplated. For example, in some embodiments, the catch-and-stop configuration can be reversed. For example, the connection interface of the catheter hub core 141 can include one or more outwardly directed catches mounted on resilient arms (which might be configured to deflect radially inwardly) and the connection interface 282 of the catheter connection hub 145 can include an outwardly directed groove into which the catches can be received and/or may include proximal and distal stops between which the catches of the core 141 are retained.

Stated otherwise, the catheter connection hub 145 can include a connection interface 174 configured to selectively couple the catheter connection hub 145 to the housing 152 and selectively decouple the catheter connection hub 145 from the housing 152. For example, as previously mentioned, in the illustrated embodiment, the connection interface 174 comprises external threading 175. In other embodiments, one or more external lugs or protrusions that can suitably couple with the threading 211 of the housing 142 may be used in place of the threading 175. Any suitable connection interface 174 is contemplated.

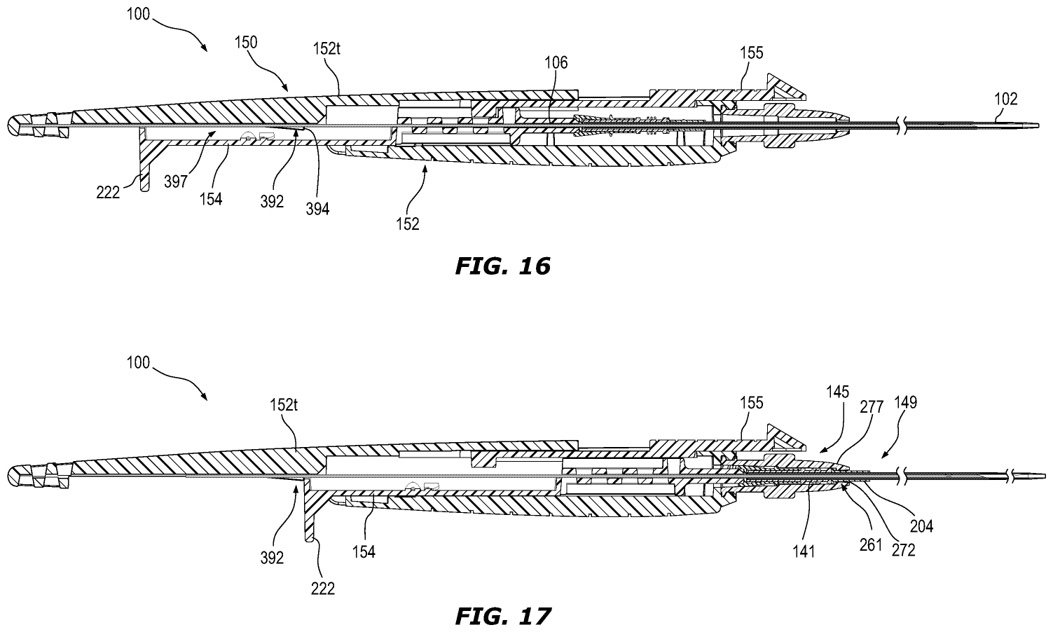

In some embodiments, the catheter connection hub 145 includes a plurality of engagement arms 184 at a distal end thereof. The engagement arms 184 can be configured to automatically couple the catheter connection hub 145 to the catheter hub core 141 during deployment of the catheter 102. In the illustrated embodiment, internally directed protrusions at the distal ends of the respective engagement arms interface with a distal of the catheter hub core 141 as the catheter hub core 141 is advanced distally. The arms 184 deflect outwardly until the catheter hub core 141 has been advanced sufficiently to permit the engagement arms 184 to automatically resiliently deflect inwardly such that the internally directed protrusions or catches 190 seat within an engagement recess of the catheter hub core 141 and thereby hold the catheter connection hub 145 in secure engagement with the catheter hub core 141. This process is depicted, e.g., in FIGS. 16-18.

In various embodiments, the catheter connection hub 145 is formed of polyurethane, such as, for example, Isoplast®, available from Lubrizol. Another illustrative example of a suitable material includes polycarbonate. Any suitable material is contemplated.

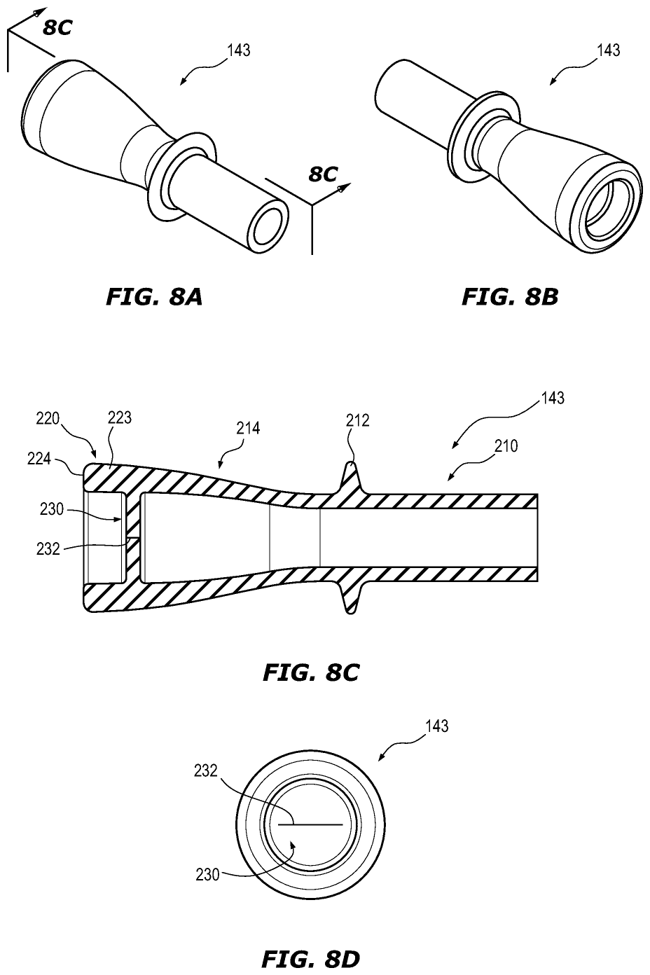

FIGS. 7A and 7B show a portion of the catheter assembly 149 in greater detail. In particular, these drawings show a catheter subassembly 153, which is initially movable relative to and connectable with the catheter connection hub 145 to form the fully assembled catheter assembly 149 (see FIG. 21). The catheter subassembly 153 may also or alternatively be referred to as an assembly. The catheter subassembly 153 can include the catheter 102 and a core assembly 144. The core assembly 144 can include the core 141 and the valving member 143 (also referred to herein as the seal member). In the illustrated embodiment, the core 141 includes a valve actuation member 202 and a strain-relief member 204. The valving member 143 and the valve actuation member 202 may operate in concert as a blood control assembly 206. The constituent components of the illustrated catheter subassembly 153 are described in greater detail with respect to FIGS. 8A-10B.

With reference to FIGS. 8A-8D, in various embodiments, the valving member 143 may be formed of a unitary component. The valving member 143 may be resiliently deformable. Any suitable material is contemplated for the valving member 143. In some embodiments, the valving member 143 comprises silicone.

In some embodiments, the valving member 143 includes an attachment portion, such as a connection sleeve 210, at a distal end thereof. The connection sleeve 210 may be configured to interface with the valve actuation member 202 to be securely attached thereto. For example, in some embodiments, an inner diameter of the connection sleeve 210 may be substantially the same as or smaller than an outer diameter of a portion of the valve actuation member 202 to which the valving member 143 is to be attached. During assembly, the connection sleeve 210 may slide over the proximal end of the valve actuation member 202 into place. Thereafter, if proximal forces are applied to the valving member 143 in an attempt to remove the valving member 143 from the valve actuation member 202, the connection sleeve 210 may stretch or otherwise be stressed so as to tightly grip the valve actuation member 202, and may operate to securely maintain a connection between the valving member 143 and the valve actuation member 202. In some embodiments, the connection sleeve 210 may directly contact the external surface of the valve actuation member 202 without any intermediary adhesive. Stated otherwise, the secure connection may be achieved without the use of adhesive, in some embodiments. The connection sleeve 210 can form a fluid-tight seal with the valve actuation member 202.

In some embodiments, the valving member 143 includes a protrusion 212 (see FIG. 8C for identification), which may operate similarly to certain seal members (e.g., those identified as seal members 843, 2043, 2143, 2743) in U.S. Pat. No. 11,065,419. The protrusion 212 may be formed as an annular ring that extends outwardly from a central body of the valving member 143. The protrusion 212 can encompass the valve actuation member 202 when the valving member 143 is joined to the valve actuation member 202, as shown in FIG. 7B.

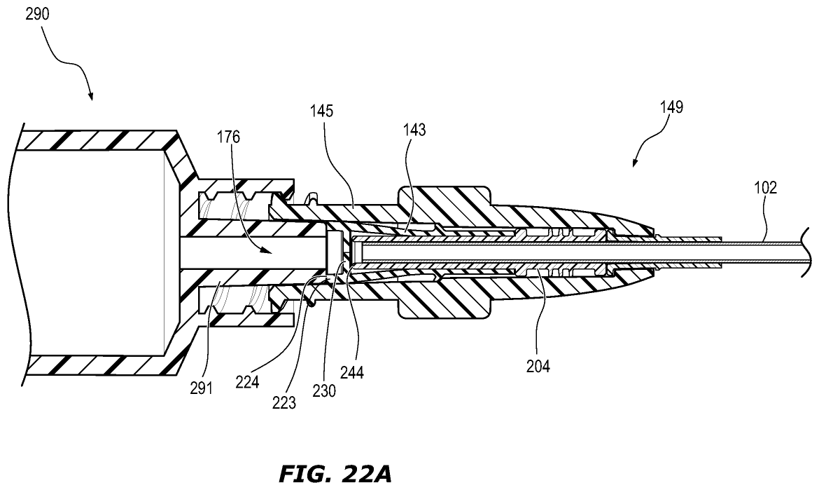

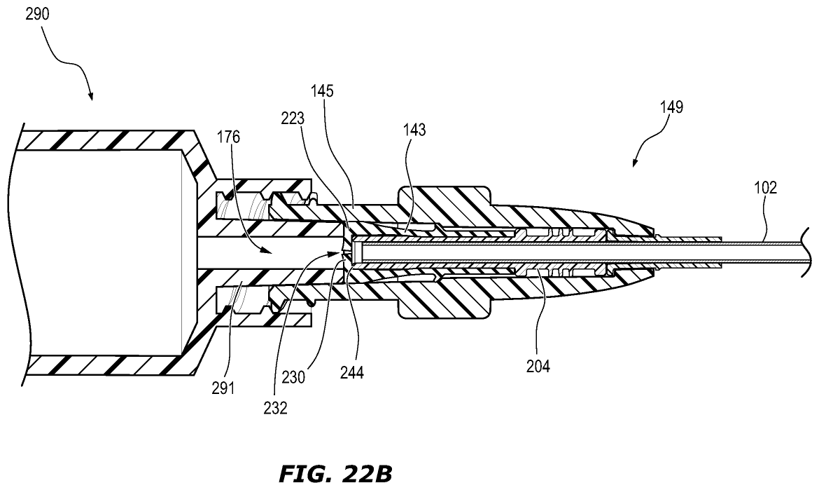

With continued reference to FIGS. 8A-8D, and with reference to FIG. 21, the protrusion 212 may be configured to interface with an inner surface of the catheter connection hub 145 and may form a fluid-tight seal therewith. For example, as shown in FIG. 21, and with further reference to FIG. 6B, when the catheter connection hub 145 is connected to the core 141, the protrusion 212 can contact an inner surface of the hub 145, which inner surface defines the cavity 176. In some embodiments, the protrusion 212 contacts the ridge 179 of the hub 145. The ridge 179 may resist distal movement of the protrusion 212 during actuation or opening of the valving member 143, which can result, for example, from distal advancement of a fluid transfer device (e.g., the male luer of a syringe) into the cavity 176 of the hub 145 (see FIGS. 22A and 22B).

In certain embodiments, the valving member 143 includes a biasing member 214. In the illustrated embodiment, the biasing member 214 is substantially conical in shape, narrowing in a proximal-to-distal direction. A distal end of the valving member 214 can be coupled with and steadily supported by the connection sleeve 210. That is, the distal end of the valving member 143 may be relatively resistant to distal movement due to the fixed relationship between the connection sleeve 210 and the underlying valve actuation member 202 and/or due to abutment of a distal end of the connection sleeve 210 against an annular protrusion, or outwardly projecting ridge or shelf, of the valve actuation member 202 (see FIG. 21). The more proximal portions of the biasing member 214 may be configured to compress, resiliently buckle, and/or otherwise deform when a distally directed force is applied to the proximal end of the valving member 143. The biasing member 214 may be viewed as a resilient bellows region that buckles and compresses under such a distally directed force and, upon removal of the force, resiliently and automatically returns to the uncompressed and undeformed or natural shape shown in FIGS. 8A-8C. In some embodiments, the resilient biasing member 214 permits the valving member 143 to repeatedly open and close as one or more fluid transfer devices are coupled with and decoupled from, respectively, the catheter assembly 149 (see FIGS. 22A and 22B).

In some embodiments, the valving member 143 comprises a proximal projection 220 that spaces a contact surface 224 from a valve 230. The proximal projection 220 can extend proximally relative to a proximal face of the valve 230. Stated otherwise, a proximal surface of the valve 230 can be recessed in a distal direction relative to the contact surface 224, or the valve 230 is distally recessed from the contact surface 224. In the illustrated embodiment, the proximal projection 220 comprises an annular extension 223. The illustrated annular extension 223 is continuous about a full periphery of the valve and is shaped substantially as a hollow cylinder. As can be seen in FIGS. 22A and 22B, the contact surface 224 can be positioned to interface with a distal tip of a fluid transfer device 290, such as the distal tip of a male luer 291 thereof, and the male luer 291 can transfer a distal displacement force to the valving member 143 via the contact surface 224.

In some instances, the proximal projection 220 can advantageously provide a safe distance between the contact surface 224 and the valve 230. For example, in some instances, the male luer of fluid transfer devices (e.g., syringes) that can be used with the catheter system 153 can vary in length and/or thickness. The male luers, when inserted, abut against at least a portion of the contact surface 224. For example, some thinner luer tips may contact only a portion (e.g., an outer ring) of the contact surface 224, while some thicker luer tips may contact substantially an entirety of the contact surface 224, and still further of the thicker luer tips may have an inner surface that has an inner diameter that is smaller than an inner diameter of the annular extension 223, or stated otherwise, may have a distal surface that contacts substantially all of the contact surface 224 while also extending radially inwardly relative to the contact surface 224. Providing a space between the contact surface 224 and the valve 230 view the proximal projection 220 can inhibit or prevent the proximal tip of the valve actuation member 202 from approximating the luer tip in a manner that might otherwise shear and/or tear the valve 230. For example, if a male luer were to have a slightly larger inner diameter than an outer diameter of the valve actuation member 202, in the absence of the proximal projection 220, the male luer might directly contact and compress the valve 230 against the proximal tip of the valve actuation member 202, which could give rise to shearing forces that could damage (e.g., cut or tear) the valve 230. The proximal projection 220 can permit use of male luers having different lengths and/or different thicknesses, such as may be due to differences arising from variations within accepted tolerance levels of a specific type of male luer (e.g., from a single manufacturer) and/or arising from differences in different types of male luers (e.g., from different manufacturers).

As shown in FIGS. 8C and 8D, the valve 230 can comprise a septum that includes a selectively sealable or closable opening, such as a slit 232. In the illustrated embodiment, the slit 232 is substantially planar, and a central axis of the valving member 143 extends therethrough. Other suitable arrangements from the sealable opening are contemplated. For example, in FIG. 24B, another slit arrangement is demonstrated. In particular, the illustrated opening includes three slits forming a tricuspid valve.

In certain embodiments, a proximal end of the valving member 143 is sized to be in radial compression when positioned within the catheter connection hub 145, such as in the position depicted in FIGS. 21 and 22A. In some instances, the radial compression can maintain the valve 230 in a tightly closed configuration. In some instances, the valve 230 is able to maintain a fluid-tight seal under extreme pressure differentials at opposite sides of the valve 230. For example, in some instances, the valve 230 can maintain a fluid-tight seal even as a user pulls proximally on a plunger of the syringe using a typical amount of force that would otherwise be used to withdraw fluid from a catheter, such as, e.g., when the syringe is positioned such as shown in FIG. 22A. The fluid-tight seal may also be sufficiently strong to prevent passage of air. That is, the fluid-tight seal may prevent passage of liquid and/or air. The valve 230 thus can be exceptionally resistant to fluid flow therethrough when in the closed state. To transition to the open state, the valve 230 is advanced distally (e.g., via application of distally directed forces to the proximal projection, e.g., via a male luer or other connector or connection interface) by an amount sufficient for the proximal tip of the valve actuation member 202 to press against the distal surface of the valve 230 and open the slit 232.

In some instances, the valve 230 is advanced distally relative to the valve actuation member 202 only a relatively small amount in order for the proximal tip of the valve actuation member 202 to sufficiently deform the valve 230 such that the slit 232 opens, thereby opening the valve 230 and establishing fluid communication between the cavity 276 of the hub 145 and the lumen 172 of the catheter 102. For example, in some embodiments, no portion of the valve actuation member 202 extends through the slit 232 in transitioning the valve 230 from the closed state to the open state. In other instances, the valve 230 may move further relative to the valve actuation member 202, and in further embodiments, at least a portion of the valve actuation member 202 may extend through the slit 232 in transitioning the valve 230 to the open state. In general, the valve 230 can be moved so as to interact with the valve actuation member 202, and can thereby provide selective communication between the lumen or cavity 176 of the hub 145 and the lumen 172 of the catheter 102 (see FIGS. 6B and 21).

In some embodiments, it can be advantageous for relatively small movement of the valve 230 to effectuate opening of the valve 230. For example, in some arrangements, a relatively smaller actuation movement can reduce the risk of shearing the valve 230 between the tip of a fluid exchange device (e.g., syringe tip) and the valve actuation member 202.

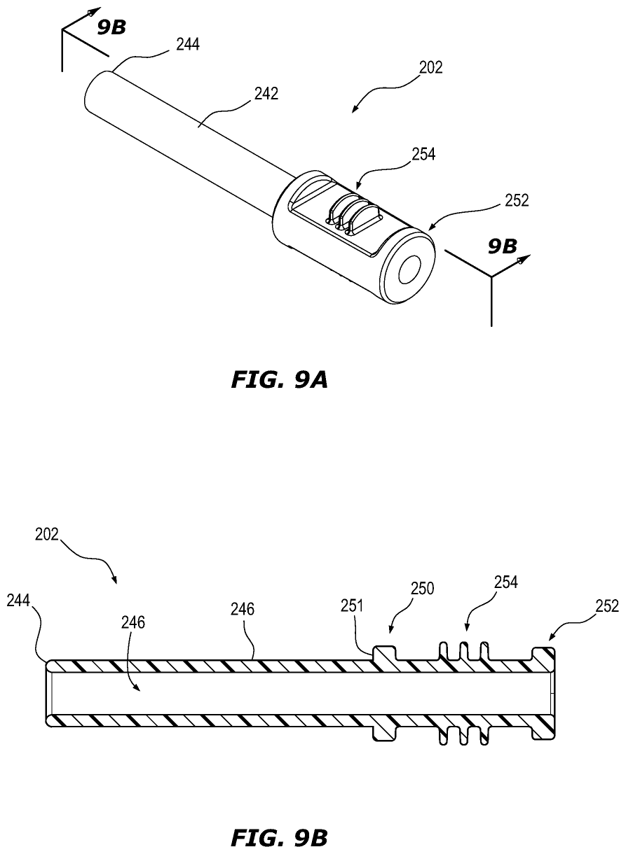

FIGS. 9A and 9B depict an embodiment of a valve actuation member 202. In the illustrated embodiment, the valve actuation member 202 includes a shaft 242, which is substantially shaped as a hollow cylinder. A proximal end of the shaft 242 includes a valve actuation tip 244, which may be rounded in some embodiments. For example, in the illustrated embodiment, the valve actuation tip 244 is substantially shaped as a hemi-torus.

The valve actuation tip 244 can be configured to urge the slit 232 of the valve 230 to separate as the valve 230 is advanced distally relative to the valve actuation tip 244. In some instances, the valve actuation tip 244 may press on and/or stretch the valve 230 and/or may advance through the slit 232 as the valve 230 is opened. The valve actuation tip 244 may be rounded, blunt, edgeless, and/or otherwise configured to interface with the valve 230 without scraping, digging into, or otherwise damaging the valve 230.

The valve actuation member 202 can include a lumen 246, which can extend through an entire longitudinal length thereof. A proximal end of the catheter 102 can be received within the lumen 246 of the valve actuation member 202. In some embodiments, as shown in FIGS. 21, 22A, and 22B, a proximal tip of the catheter 102 can be distally recessed relative to the valve actuation tip 244. It may also be said that the proximal end of the lumen 172 of the catheter 102 can be positioned within the lumen 246 of the valve actuation member 202. The valve actuation member 202 can be fixedly secured to the catheter 102 in any suitable manner. In various embodiments, the valve actuation member 202 can be overmolded onto the catheter 102 or can be adhered to the catheter 102.

In the illustrated embodiment, the valve actuation member 202 includes two outwardly projecting rings 250, 252. The proximal ring 250 includes a stop surface 251 that can interface with a distal end of the connection sleeve 210 of the valving member 143, as previously discussed. In some embodiments, a distal face of the distal ring 250 can abut a proximal end of the strain-relief member 204, as shown, e.g., in FIG. 7B.

In some embodiments, the valve actuation member 202 includes a plurality of fins 254. In the illustrated embodiment, the actuation member 202 includes two sets of fins 254 that extend in opposite directions. Each set includes three equally spaced apart fins 254. In some embodiments, the fins 254 and/or the rings 250, 252 can assist in centering the valve actuation member 202 within the catheter connection hub 145, such as during advancement of the valve actuation member 202 into the catheter connection hub 145, as shown in FIGS. 17-19. The fins 254 and/or the rings 250, 252 can stabilize, wedge, and/or secure a distal end of the valve actuation member 202 within the catheter connection hub 145 when the actuation member 202 is being advanced distally and when it is fully seated within the catheter connection hub 145 in a fully assembled state. This arrangement can help maintain a central longitudinal axis of the catheter 102 and of the valve actuation member 202 in alignment with a central longitudinal axis of the system 100 during deployment (see FIGS. 17-19) and/or can maintain such an alignment with a central longitudinal axis of a fluid transfer device (e.g., syringe) when such is being coupled with the catheter connection hub 145 (see FIGS. 22A and 22B).

The valve actuation member 202 can be formed of a stiff or rigid material. Any suitable material is contemplated. In some embodiments, the valve actuation member 202 comprises a thermoplastic polyurethane.

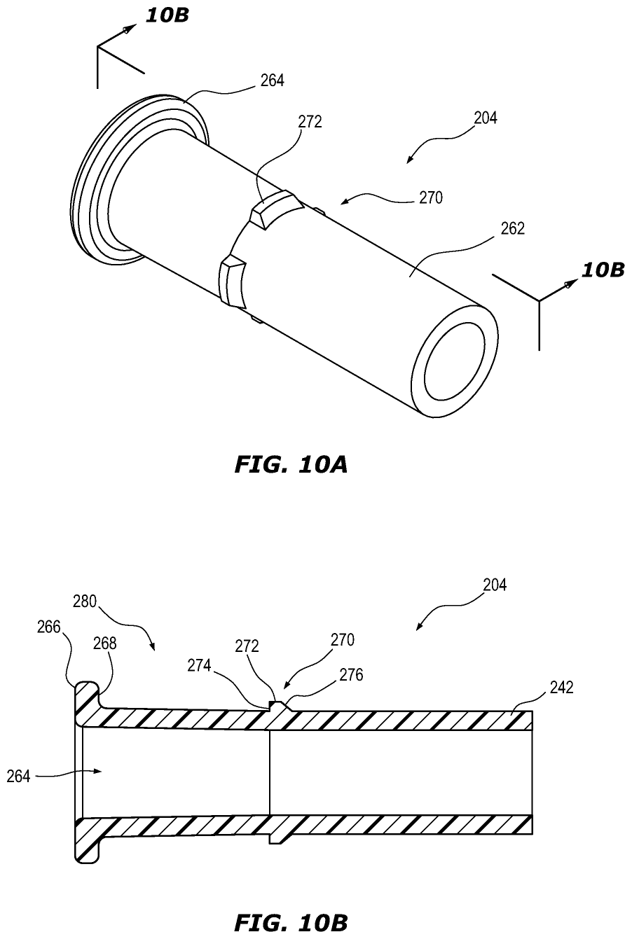

FIGS. 10A and 10B depict an embodiment of a strain-relief member 204. In the illustrated embodiment, the strain-relief member 204 includes a shaft 262, which is substantially shaped as a hollow cylinder. The strain-relief member 204 defines a lumen 264 which can be sized to receive the catheter 102. The strain-relief member 204 may be fixedly secured to the catheter 102 in any suitable manner, such as by overmolding or adhesive.

The strain-relief member 204 may, for example, be more flexible than the valve actuation member 202 and less flexible than the catheter 102. The strain-relief member 204 may be formed of any suitable material. For example, in some embodiments, the strain-relief member 204 comprises a polyurethane, and may have a hardness within a range of, for example, 97 shore A to 55 shore D.

In the illustrated embodiment, the strain-relief member 204 includes an outwardly projecting ring 266. In some embodiments, a proximal face of the ring 266 can abut a distal face of the distal ring 252 of the valve actuation member 202, as depicted, e.g., in FIG. 7B.

The ring 266 can also be referred to herein as a stop element. The stop element 266 can include a stop surface 268 that is configured to interface with the catches of the arms of the hub 145 to prevent distal movement of the strain-relief member 204 relative to the hub 145 after engagement has been established, as previously discussed.

The strain-relief member 204 can also include a retention lip 270, which may also be referred to as a stop element. The illustrated retention lip 270 is formed of a plurality of separate lip elements 272 that extend about a perimeter of the strain-relief member 204. A proximal face of each lip element 272 includes a stop surface 274 that is configured to inhibit proximal movement of the catheter 102 relative to the hub 145, once the hub 145 has been connected to the catheter 102 via the arms, as previously discussed.

The distal face of each lip can include a ramp 276, which can facilitate outward deflection of the catches of the arms of the hub 145 as the catheter subassembly 153 is advanced distally into connection with the hub 145.

The strain-relief member 204 may be said to include a connection region 280 that is responsible for establishing a connection with the hub 145 and thereafter retaining the strain-relief member 204, and thereby the rest of the catheter subassembly 153 that is attached thereto, in connection with the hub 145. The connection region 280 can include the ring 266, and particularly the stop surface 268 of the ring 266, at a proximal end thereof, and can further include the retention lip 270 at the distal end thereof. As previously discussed, the retention lip 270 can interface with the catches of the arms of the hub 145 to deflect the arms 184 as the retention lip 270 passes through the catches 190. Thereafter, the stop surfaces 268, 274 interface with the surfaces 192, 194, respectively, of the catches 190 to maintain the hub 145 in connection with the strain-relief member 204. The hub 145 is thereby directly secured to the strain-relief member 204, and, indirectly via the strain-relief member 204, is secured to the remainder of the catheter subassembly 153. In other embodiments, the connection region 280 may be configured in other manners and/or may be present at other portions of the catheter subassembly 153. For example, in some embodiments, the valve actuation member 202, rather than the strain relief member 104, may define the connection region 280. In further embodiments, the strain-relief member 104 may be omitted.

FIGS. 2 and 14-21 depict various stages of operation of the system 100. Stated otherwise, FIGS. 2 and 14-21 depict various stages or steps of illustrative methods, such as methods of using the system 100. Accordingly, the following discussion of these drawings disclose both operational details of embodiments of the system 100, as well as illustrative methods, including methods that specifically employ embodiments of the system 100.

FIGS. 2, 14, and 15 depict the system 100 in an undeployed, pre-use, as-packaged, or initial state. For example, the system 100 is shown in a state in which the system 100 may be sterilized, packaged, delivered to a user, and/or removed from packaging by the user. Stated otherwise, the user may, in some instances remove the system 100 from packaging in substantially the illustrated configuration.

In some embodiments, the system 100 includes a cap (not shown), which can cover the distal tip of the needle 104 to prevent inadvertent sticks prior to intended use. Any suitable mechanisms may also be employed to maintain the upper actuator 155 and the lower actuator 222 in their respective retracted states. For example, in some embodiments, the cap and/or a separate spacer or stop element (not shown) can be configured to maintain the upper actuator 155 in the fully retracted or undeployed orientation. Further, in some embodiments, a separate cap, spacer, or stop element and/or packaging for the system 100 can prevent actuation of the lower actuator, such as during transport.

The general arrangement of and relationships between the catheter 102, the needle 104, and the stiffener 106 have previously been described. As shown in FIG. 15B, in the initial operational state, a distal end of the needle 104 can extend past a distal end of the catheter 102. The distal end of the catheter 102 can include a catching region 191 that can interface with a distal tip of the stiffener 106. As described further below, during deployment, the stiffener 106 can be advanced distally relative to the needle 104, which can press on the catching region 191 of the catheter 102. The stiffener 106 can serve to push the distal end of the catheter 102 off of and away from the distal end of the needle 104, and can assist in advancing the catheter 102 distally relative to the needle 104. For example, proximal portions of the catheter 102 may be seen as being pulled into the vasculature via the distal tip of the stiffener 106 as it remains in engagement with and pushes distally against the catching region 191.

As previously mentioned, and as shown in FIGS. 2, 14, and 15B, in the initial or retracted state, the needle 104 can extend through an entirety of an interior of the stiffener 106. The proximal end of the needle 104 is fixedly secured to the housing 152. At least a distal portion of the stiffener 106 can be positioned within a lumen defined by the catheter 102. Stated otherwise, at least a portion of the stiffener 106 can be positioned between an inner surface defined by the catheter 102 and an outer surface defined by the needle 104. The catheter 102, stiffener 106, and needle 104 can be concentrically oriented, share a common longitudinal axis, and/or stated otherwise, can be in a nested configuration, as shown. The stiffener 106 can be translatable relative to the needle 104 and, at least initially, is translatable in a distal direction.

With reference to FIG. 15B, when the system 100 is in the undeployed state, a port 180 through the sidewall of the needle 104 and a port 182 through the sidewall of the stiffener 106 (which may also be referred to as a flash port 182) can be aligned to define a passageway 186 for the passage of blood. In particular, the passageway 186 may permit a flash of blood to pass from the lumen of the needle 104 into an elongated annular lumen 188 defined by the inner surface of the catheter 102 and the outer surface of the stiffener 106. That is, blood received into the needle tip and can pass through the passageway 186 and can flow proximally through the catheter 102 to provide a visual indication to the user of a desired placement of the needle 104 (e.g., the blood may be visible through a clear, translucent, or not fully opaque catheter 102). Stated otherwise, the passageway 186 and the lumen 188 can provide a channel through which a flash of blood can pass to indicate that the distal end of the needle 104 has entered a blood vessel, such as previously described. The flash port 182 is also identified in FIG. 13.

With reference to FIG. 2, in the pre-use configuration, each of the upper and lower actuators 155, 222 is in a fully retracted position. Stated otherwise, each of the upper and lower actuators 155, 222 is at a proximal-most or fully rearward position. Accordingly, the stiffener hub 154 is also in a fully retracted position.

In the illustrated embodiment, the upper actuator 155 does not initially engage the stiffener hub 154 when both components are in their retracted orientations. In particular, as can be seen in FIG. 2 (with reference also to FIGS. 11 and 13 for numbering), a small space or gap is present between the engagement face 326 of the engagement protrusion 325 of the upper actuator 155 and the engagement face 381 of the stiffener hub 154. Accordingly, the upper actuator 155 is advanced forwardly a very short distance to initially engage the stiffener hub 154.

In other embodiments, the engagement faces 326, 381 of the upper actuator 155 and the stiffener hub 154 are in abutting contact in the pre-use state of the system 100, such that forward movement of the upper actuator 155 immediately achieves concurrent forward movement of the stiffener hub 154. In some instances, the presence and/or size of any initial gap between the engagement faces 326, 381 can vary from system 100 to system 100 within an acceptable tolerance range, such that no forward movement or only slight movement of the upper actuator 155 is required prior to the upper actuator 155 engaging the stiffener hub 154 for any of the systems 100 manufactured within specification.

With reference to FIGS. 2, 14, and 15A, as previously noted, the distal tip 334 of the stiffener hub 154 can interface with the valving member 143 to apply deployment forces to the catheter subassembly 153 in the distal direction. In the illustrated embodiment, the distal tip 334 of the stiffener hub 154 engages proximal portions of the valving member 143 of the catheter subassembly 153 while the system 100 is in the pre-use or pre-deployment state. In some instances, it can be desirable to ensure that the stiffener hub 154 engages the catheter subassembly 153 in this initial state of the system 100 to ensure that the stiffener 106 and catheter 102 move substantially in unison immediately upon actuation of the stiffener hub 154. That is, the stiffener hub 154 immediately transfers force to the catheter subassembly 153 such that both components or systems move forward in unison. Such an arrangement can alleviate strain forces along the length of the catheter 106 that might otherwise arise in the absence of the stiffener hub 154 pushing the catheter hub core 141 forward.

For example, advancement of the stiffener 106 can cause the distal tip of the stiffener 106 to push forwardly on the distal tip of the catheter 102. This not only causes the distal tip of the catheter 102 to move forward, but also draws the remainder of the catheter 102 forward as well, due to stresses exerted along the length of the catheter 102. Should forward movement of the catheter hub core 141 be impeded, strain along the length of the catheter 102 can increase.

By urging the catheter subassembly 153 forward, the stiffener hub 154 alleviates stresses along at least a portion of the length of the catheter 102. This stress alleviation can be particularly pronounced, and particularly useful, at latter stages of the catheter deployment for certain embodiments, where increased force may need to be provided to the catheter hub core 141 to spread open the resilient arms at the distal end of the catheter connection hub 145 during coupling of the catheter hub core 141 to the catheter connection hub 145 (in manners such as previously discussed). In such instances, all or substantially all force required to couple the catheter hub core 141 to the catheter connection hub 145 can be provided directly to the catheter hub core 141 by the stiffener hub 154.

The strain relief provided to the catheter 102 by the interfacing of the stiffener hub 154 with the proximal end of the catheter subassembly 153 can be explained in other terms. For example, by ensuring a direct coupling between the stiffener hub 154 and the catheter subassembly 153 exists in the initial, pre-use state of the system 100, both the proximal ends and distal ends of the catheter 102 and the stiffener 106 move forward at the same rate. Stated otherwise, a length of the catheter 102 and a length of the stiffener 106 are each substantially constant throughout deployment, and further, the catheter 102 and the stiffener 106 move forward in unison.

In other embodiments, a space or gap may be present between the distal tip 334 of the stiffener hub 154 and the valving member 143 of the catheter subassembly 153 when the system is in the initial or pre-use orientation, and potentially through at least some of the subsequent phases of deployment. For example, a small gap may be present due to manufacturing tolerances or the like. As a further example, in some instances, a small gap may be present to limit an amount of compression or overall strain experienced by the valving member 143 during a full life cycle of the system 100. In certain of such instances, the valving member 143 may not be directly contacted during translation of the catheter subassembly 153 forward unless and until sufficient strain on the catheter 102, as the stiffener 106 advances the catheter 102 into the vasculature, slightly elongates the catheter 102 such that the proximal end of the catheter subassembly 153 comes into contact with the distal tip of the stiffener hub 154. For example, in certain of such embodiments, the catheter hub core 141 and valving member 143 may be pulled distally by the catheter body 102 up until the catheter hub core 141 comes into contact with the resilient arms of the catheter connection hub 145. Due to the increased resistance to distal movement provided by the catheter connection hub 145, the catheter body 102 may elongate as the stiffener 106 is urged distally to the point where the stiffener hub 154 engages the valving member 143. At this point, the stiffener hub 154 can directly push on the valving member 143, which can transfer forces from the stiffener hub 154 to the catheter hub core 141, thereby supplementing the distal forces on the catheter hub core 141 that are also provided to the catheter hub core 141 through an indirect path—specifically, the stiffener hub 154 urges the stiffener 106 forward, which urges the distal tip of the catheter 106 forward, which pulls forward the proximal end of the catheter 102 and the catheter hub core 141 to which it is attached.

With reference to FIG. 15A, in the illustrated embodiment, the distal tip 334 of the stiffener hub 154 directly contacts the proximal side of the valve 230 of the valving member 143 in the initial or pre-use state and/or during subsequent advancement of the catheter subassembly 153. A proximal side of the valve 230 can thereby be pressed against the proximal tip 244 of the valve actuation member 202 such that the valve 230 transfers displacement forces to the valve actuation member 202 and the catheter 102 to which it is fixedly secured. As previously noted, in the illustrated embodiment, the distal tip 334 of the stiffener hub 154 includes a flat end and a conical surface extending proximally therefrom. In other embodiments, the distal tip 334 may be rounded (e.g., may include a toroidal surface).

In other embodiments, the distal tip 334 of the stiffener hub 154 and/or the proximal tip 244 of the valve actuation member 202 may be spaced from the valve 230 during a deployment event. For example, in some embodiments, the proximal annular extension 223 of the valving member 143 may be sufficiently long and/or thick to prevent contact between the distal tip 334 of the stiffener hub 154 and the proximal face of the valve 230 throughout at least a portion of a deployment event.

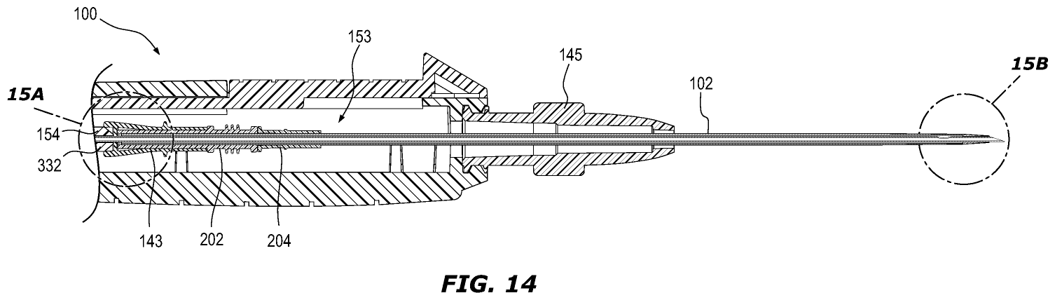

With reference again to FIGS. 2 and 14, in the illustrated embodiment, when the system 100 is in the pre-use or undeployed state, the catheter hub core 141 is spaced from the catheter connection hub 145. In particular, the catheter hub core 141 is entirely separate from the catheter connection hub 145, is not in contact therewith, and is distanced from the catheter connection hub 145 by a significant length. The catheter hub core 141 is positioned rearward of or proximal to the catheter connection hub 145. The catheter hub core 141 is positioned at an interior of the housing 142, or stated otherwise, is fully received within the cavity 202 of the housing 142. The housing may 142 may be said to encompass, encircle, or enclose the catheter hub core 141. Moreover, in the illustrated embodiment, no portion of the catheter hub core 141 is encompassed, encircled by, or enclosed by the catheter connection hub 145 when the system 100 is in the pre-deployed state.

In contrast, in the illustrated embodiment, the catheter connection hub 145 is coupled to a distal end of the housing 152 in manners such as previously disclosed. Accordingly, the catheter connection hub 145 is connected to the housing 152 via a connection interface 210. With the exception of the coupling interface 210, substantially an entire exterior surface of the catheter connection hub 145 is at an exterior of the housing 142. An interior of the catheter connection hub 145 is, however, in fluid communication with the cavity 202 of the housing 142. Further, in the illustrated embodiment, with the exception of the proximal portion of the catheter connection hub 145 that defines the connection interface 210, a substantial portion or most of the catheter connection hub 145 extends distally away from the housing 142 and is external to the housing 142.

In the initial state of the system 100, the catheter hub core 141 is free to translate within the housing 141 in manners such as previously disclosed (e.g., slide longitudinally while remaining rotationally locked). In contrast, the catheter connection hub 145 is in a selectively fixed relationship relative to the housing 142.

As further discussed below, when the system 100 is in the undeployed state, the distal end of the system 100 (e.g., the distal tips of the needle 104, the catheter 102, and stiffener 106) can be advanced through the skin of a patient, thereby establishing an insertion site of the skin, and at least a distal tip of the needle 104 can further be advanced into a vessel of the patient, thereby establishing a vessel insertion site. In some instances, deployment of the system 100 begins after only the tip of the needle 104 has been advanced into the vessel. In other instances, the distal tip of the catheter 102—and, in further instances, the distal tip of the stiffener 106 as well—likewise enters into the lumen of the vessel through the vessel insertion site by a relatively small amount while the system 100 is in the undeployed state.

Once a suitable portion of the distal end of the system 100 is within the lumen of the vessel, as indicated by a flash of blood in manners such as previously disclosed, the system 100 can then be actuated or deployed to insert the catheter 102 into the vessel and thereafter advance the catheter 102 to a final or maximum depth within the vessel (e.g., where only the distal tip of the needle 104 was initially inserted into the vessel lumen), or to advance the catheter 102 to the final depth within the vessel (e.g., where at least the tip of the catheter 102 was also initially inserted into the vessel lumen).

FIG. 16 depicts the system 100 in a subsequent operational state. In particular, the system 100 has been partially deployed or deployed an intermediate amount via the upper actuator 155. A range of partial or intermediate deployments of the system 100 are possible via the actuator 155. In the illustrated stage, the upper actuator 155 has been advanced to its maximum forward position, which has resulted in forward movement of the stiffener hub 154, and hence concurrent forward movement of the stiffener 106 and the catheter 102 through a first distance. In some embodiments, the first distance can be selected to ensure that the stiffener 106 and the catheter 102 are advanced through the lumen of the vessel to a depth sufficient to maintain the catheter 102 and the stiffener 106 within the vessel for a temporary period prior to final advancement of the catheter 102 and the stiffener 106 to the maximum deployed depth via the lower actuator 222.

Stated another way, in the operational phase depicted in FIG. 16, the upper actuator 155 has been advanced along an entirety of the open track defined by the housing 152, and thus along a predetermined distance. This forward movement of the upper actuator 155 pulls the stiffener hub 154 forward by a corresponding or roughly corresponding amount. In particular, when the upper actuator 155 is moved from its fully retracted position to its fully advanced position, as shown, the stiffener hub 154 also moves a predetermined distance, which may be the same as or substantially the same as the predetermined distance travelled by the upper actuator 155.

The first deployment distance traveled by the stiffener hub 154 may also be referred to as a stabilization, anchoring, and/or retention distance, as advancing the catheter 102 into the vessel to this distance can help to ensure that the catheter 102 remains positioned within the vessel for at least an intermediate period. The intermediate period can begin after the initial deployment phase achieved via the upper actuator 155 has ceased and can end once actuation of the lower actuator 222 to achieve a final deployment of the system 100 begins.

In some instances, the upper actuator 155 can conveniently be advanced forwardly in a variety of ways using a single finger (e.g., the index finger) of a hand while that same hand is holding the handle 150. In the illustrated embodiment, the upper actuator 155 is at the forward end of the handle 150, which can facilitate this form of actuation.

In FIG. 16, the upper actuator 155 has been advanced fully to its distalmost, advanced position. The upper actuator 155 has pulled the stiffener hub 154 forward by the same amount, but the stiffener hub 154 remains in only a partially actuated or partially deployed state. At this operational state, the stop 392 remains within the cavity 394 of the stiffener hub 154 and thus does not inhibit movement of the stiffener hub 154. Accordingly, the additional forces that come into play when the longitudinally elongated portion of stiffener hub 154 and/or a proximal portion of the upper housing element 152t are deflected away from each other by interaction of a proximal end of the stiffener hub 154 with the the ramped surfaces of the stop 392, as discussed further below, do not have any bearing on deployment of the upper actuator 155.

In the illustrated embodiment, when the system 100 is in the intermediate state, the stop 392 defined by the upper housing element 152t is positioned within a cavity 397 defined by the stiffener hub 154. The stop 392 does not restrict distal or proximal movement of the stiffener hub 154 when positioned within the cavity 397. (It is noted that the stop 392 is similarly within the cavity 397 in the initial state depicted in FIG. 2.)

FIG. 17 shows a subsequent stage in which the proximal end of the stiffener hub 154 has been advanced just past the stop 392. In the illustrated embodiment, the upper housing element 152t and the stiffener hub 154 are formed of flexible (e.g., resiliently flexible) material and also define long moment arms in the longitudinal direction, and one or both of these components can readily bend by a small amount—away from the other or away from each other—as the proximal end of the stiffener hub 154 is advanced over the ramped surfaces 393 of the stop 392 (see also FIG. 4). Due to the resiliency of the materials used, the housing element 152t and/or the elongated proximal stem of the stiffener hub 154 have resiliently returned, or snapped back, into a substantially parallel orientation relative to each other. In some instances, only a small sound or no discernable sound may be made and/or only a slight difference in pushing force may be detectable by the user when this snap back occurs so as to avoid any confusion as to when the system 100 has been fully deployed to the point that catheter hub core 141 audibly snaps into the catheter connection hub 145. Indeed, in the illustrated embodiment, all interactions between the stop 392 and the stiffener hub 154 is completed before the catheter hub core 141 is securely engaged by the flexible gripping arms of the catheter connection hub 145.

Once the stiffener hub 154 has been advanced distally to the position shown in FIG. 17, the stop 392 prevents the stiffener hub 154 from being retracted or moved proximally past the stop (unless extra and deliberate force for achieving such a retraction are exercised, such as to deliberately reset the device, as discussed below). Stated otherwise, the stop 392 can inhibit or prevent, under normal usage, the proximal retraction of the stiffener hub 154 once the distal position depicted in FIG. 17 has been reached.

In the operational stage depicted in FIG. 17, the lip elements 272 of the strain-relief member 204 have caused the resilient arms 277 of the catheter connection hub 145 to spread apart as the catheter hub core 141 has been moved distally. The distal movement has not yet been sufficient, however, for the resilient arms 277 to resiliently return to a resting state, or snap back in an inward direction, into engagement with the engagement recess 261, as previously described.

FIG. 18 illustrates that the stiffener hub 154 has been advanced distally by a small additional amount to permit the resilient arms 277 to engage the engagement recess 261 of the catheter hub core 141. In some instances, the increase in force required to push past the inner protrusions of the resilient arms 277 can be perceptible, relative to the forces used during all prior phases of deployment, so as to provide tactile feedback to the user regarding the status of the construction of the hub assembly 149. In some instances, the release of energy and/or click or other sound made by the resilient arms 277 as they automatically secure to the catheter hub core 141 may provide further or other feedback to the user regarding the assembly status of the hub assembly 149. A comparison between FIGS. 17 and 18 shows that in the illustrated embodiment, a small additional distal movement of the stiffener hub 154 past the stop 392 is permitted and, indeed, may be needed in some instances, to fully assemble the hub assembly 149.

In like manner, the stiffener hub 154 may be translatable by the small amount in the proximal direction, such that there is a small amount of longitudinal play in the stiffener hub 154, but substantial proximal movement of the stiffener hub 154 relative to the housing 152 is inhibited or prevented by the stop 392.

Stated otherwise, FIG. 18 depicts the system 100 in a fully deployed state, or deployment by a complete or maximum amount. The final amount of actuation has been achieved via the lower actuator 222. A range of partial or intermediate deployments of the system 100 are also possible via the lower actuator 222. However, in the illustrated stage, the lower actuator 222 has been advanced to its maximum forward position, which has resulted in forward movement of the stiffener hub 154, and hence concurrent forward movement of the stiffener 106 and the catheter 102 through an additional second distance. Accordingly, the system 100—and, specifically, the stiffener hub 154 of the system 100—has been actuated through a total distance that is the sum of the first distance mentioned above (i.e., resulting from the indirect actuation of the stiffener hub 154 via the upper actuator 155) and the second distance mentioned above (i.e., resulting from the direct actuation of the stiffener hub 154 via the lower actuator 222).

Stated otherwise, after initial actuation of the system 100 via the upper actuator 155, the lower actuator 222 can be advanced the remainder of an available forward path to finish deploying the catheter 102/stiffener 106 combination over the needle 104.

In the illustrated embodiment, no further forward movement of the upper actuator 155 occurs during direct actuation of the lower actuator 222. Stated otherwise, the upper actuator 155 may disengage from the stiffener hub 154 and remain stationary relative to the housing 152 during the further forward advancement of the stiffener hub 154.

The forward path traveled by the stiffener hub 154 can be delimited by the catheter connection hub 145. Stated otherwise, coupling of the catheter hub core 141 with the catheter connection hub 145 can terminate forward advancement of the stiffener hub 154. In particular, in the illustrated embodiment, the lower actuator 222 is urged (e.g., pressed) forward to directly advance the stiffener hub 154 forward. As previously discussed, throughout either a portion of or an entirety of this forward advancement of the stiffener hub 154, the stiffener hub 154 can engage and press on a proximal end of the catheter hub core 141, thus urging the catheter hub core 141 forwardly. The user can be provided with a tactile feedback that the catheter hub core 141 has begun engaging the resilient arms of the catheter connection hub 145 as resistance to forward movement of stiffener hub 154 can increase. Ultimately, the catheter hub core 141 is advanced distally by a sufficient amount to permit the deflected engagement arms of the catheter connection hub 145 to snap into a groove or channel of the catheter hub core 141 and firmly hold the catheter hub core 141, as described more fully above. Because the catheter connection hub 145 is securely connected to the housing 152 and the catheter hub core 141 is securely connected to the catheter connection hub 145 at this point, the user can be prevented from advancing the stiffener hub 154 any further relative to the housing 152. This significant resistance or complete opposition to further advancement of the stiffener hub 154 relative to the housing 152 can provide further tactile feedback to the user, this time indicating that deployment is complete and the catheter assembly 149 is fully assembled.

In some embodiments, the user may also receive auditory feedback that deployment is complete. For example, the catheter connection hub 145 and/or the catheter hub core 141 may individually or in cooperation generate an auditory signal upon coupling. In the illustrated embodiment, connection of the catheter connection hub 145 to the catheter hub core 141 generates an audible “click,” indicating that coupling is complete.

In other or further instances, the forward path can be delimited by direct contact between the stiffener hub 154 and the lower housing element 152b. In any event, the stiffener hub 154 may cooperate directly or indirectly with the housing 152 to delimit forward movement of the stiffener hub 154.

As previously discussed, in some instances, the lower actuator 222 can conveniently be advanced forwardly by one hand of a user while the user holds the housing 152 with the other hand. In some instances, it may be convenient or otherwise advantageous for the lower actuator 222 to be positioned rearward of the lower housing element 152b, generally rearward of the upper actuator 155, and/or extend downward relative to the housing 152 generally. In some instances, such an arrangement can yield a compact system 100, as the lower actuator 222 does not extend significantly beyond lower profile of the lower housing element 152b. Nevertheless, the illustrated actuator 222 is sufficiently large to be readily gripped and/or readily pushed to deploy, or further deploy, the system 100. In some instances, such a significant rearward location of the lower actuator 222 can permit the handle 150 to be positioned close to the skin of the patient, which can permit shallow insertion angles.

With further reference to FIGS. 2 and 16-18, a two-actuator system 100 and two-phase deployment process, as just described can be advantageous in some instances. The first stage of actuation (e.g., an index finger flick or other advancement of the upper actuator 155) can assist in an initial capture the vessel, and the second stage of actuation can then advance the catheter 102 to its final or fully inserted position.

This may be particularly useful in deep vein placements of the catheter 102. In such placements, a practitioner may use, e.g., the nondominant hand to press against the skin above the vessel to provide tension to the region and assist in positioning the vessel and/or aligning the system 100 with the same. The other (e.g., dominant) hand can grip the system 100 (or any desired portion thereof, such as the handle 150), and advance the full system 100 forward to introduce the needle tip (and potentially the catheter tip as well, at this stage) into the vessel until a flash of blood is seen.

Removal of the non-dominant hand from the skin of the patient at this point, prior to deployment of the catheter 102 into the vessel over the needle 104, could allow sufficient shifting of the vessel and surrounding tissues, or otherwise destabilize the region and/or allow inadvertent movement of the dominant hand and the system 100 it is holding relative to the region, in a manner that the needle 104 and catheter 102 inadvertently emerge from the vessel. To prevent this, after placement of the needle tip in the vessel, it can be desirable to advance the catheter 102 (e.g., via the sheathing cannula 106) into the vessel to, e.g., at least ½ inch or so to prevent inadvertent removal of the catheter 102 from the vessel when the nondominant hand is removed in order to actuate the lower actuator 222 thereby. This is accomplished by advancing the upper actuator 155 forward while both hands maintain steady positioning, such as pressure on the patient with the nondominant hand and gripping of the system 100 with the dominant hand. After initial capture of the vessel in this manner and removal of the nondominant hand from the patient's skin, the nondominant hand can then be used to advance the lower actuator 222 to finish advancing the catheter 102 into the patient to the target depth, or stated otherwise, to the fully deployed position.

Capture of the vessel in the foregoing manner may be referred to in a variety of ways. For example such vessel capture may also be referred to as stabilizing or anchoring the system 100 relative to the vessel. That is, the catheter 102 is desirably advanced to a position within the vessel that will permit retention of the catheter 102 within the vessel, despite small or inadvertent relative movements between the vessel and the system 100. Accordingly, the initial distance to which the catheter 102 is advanced over the needle within the vessel may be referred to as a capture, stabilization, anchoring, or retention distance. Such advancement of the catheter 102 is preparatory to the final deployment of the catheter 102 to its final position within the vessel, which may also be referred to as the indwelling, fully advanced, or resident position, etc.

In some instances, placement of the lower actuator 222 below the handle 150 allows the dominant hand to maintain continuous contact with the handle 150 throughout both the initial introduction of the catheter 102 into the vessel and subsequent actuation of the system 100 for further deployment the catheter 102 to a final depth within the vessel. For example, by gripping the handle 150 with the dominant hand, the fingers may wrap around the housing 152, but not extend over the pathway along which the lower actuator 222, or more generally, the stiffener hub 154, is slid. As the dominant hand grips the housing 152, with the index finger on one side and the other fingers on the other, the nondominant hand can engage the lower actuator 222 and move it forward between the thumb and fingers of the dominant hand, without disrupting placement of the thumb and fingers of the dominant hand. In other instances, the roles of the dominant and nondominant hands can be reversed.

In the illustrated embodiment, the upper actuator 155 captures or engages the stiffener hub 154 when advanced in the distal direction, but not when pulled in the proximal direction, which is depicted in FIG. 19. The stiffener hub 154 does not capture or engage the upper actuator 155 when moved in the forward direction, but the stiffener hub 154 may capture or engage the upper actuator 155 if the stiffener hub 154 is pulled in the rearward direction (if the upper actuator 155 has previously been advanced distally).

With reference again to FIG. 2, in some instances, a user may forego using the upper actuator 155 and may opt instead to use only the lower actuator 222 to deploy the catheter 102. That is, the lower actuator 222 may be moved the full deployment distance directly, or without use of the upper actuator 155, which would yield a configuration such as shown in FIG. 19. That is, by using solely the lower actuator 222, the user could move directly from the configuration of FIG. 2 to that shown in FIG. 19 (e.g., where the upper actuator 155 remains substantially unmoved relative to the housing 152). In the illustrated arrangement, due to a lack of interaction between the stiffener hub 154 and the upper actuator 155 during forward movement of only the stiffener hub 154 from the position depicted in FIG. 2, the upper actuator 155 may remain in its initial position during such a deployment. Thus, when the system 100 is in the fully deployed state, the upper actuator 155 may be positioned in a fully retracted state rather than the fully advanced state. With reference to 17, then, upon full deployment of the system 100 in this manner, the actuator 155 would be positioned in its leftmost (i.e., proximal most), rather than rightmost (i.e., distal most), orientation.

In some instances, a practitioner may opt to use such a one-stage actuation in contexts such as peripheral placements. For example, a practitioner may, in some instances, prefer to use only the lower actuator 222 to deploy the catheter 102 if vessel access is relatively straightforward. The practitioner may insert the tip of the needle 104 (and potentially the tip of the catheter) into the vessel a desired initial amount (e.g., while the system 100 is in the undeployed stated) without using the other hand for tensioning/positioning purposes, due to the relative accessibility (e.g., due to shallow position) of peripheral vessels. Once the system 100 has been inserted into the vessel to an initial depth (which may also be referred to as an introduction depth), the practitioner may then slide only the lower actuator 222 to advance the catheter 102 into the vessel to the final or indwelling depth.

Accordingly, the system 100 can be usable in two different deployment modes—i.e., in a two-phase deployment mode or a one-phase deployment mode. A user thus can select which mode to use based on preference, type of vessel being accessed, etc.

Alternatively, FIG. 19 may depict a stage of a two-actuator deployment method operation in which the upper actuator 155, after having been fully deployed distally, has been retracted proximally away from the catheter connection hub 145. In some instances, moving the upper actuator 155 in this manner can facilitate removal of the catheter connection hub 145 from the housing 152. This view also demonstrates that the upper actuator 155 does not engage the stiffener hub 154 when moved rearwardly, and thus the stiffener hub 154 remains in its forward, fully deployed position.

FIGS. 20 and 21 depict a later stage of operation in which the insertion assembly 109 has been removed from the catheter assembly 149 while the catheter assembly 149 is held in place relative to the patient, with the catheter 102 positioned at its indwelling position with the vessel. For example, in some instances, the user can hold the catheter assembly 149 steady or stationary relative to the patient with one hand while withdrawing the insertion assembly 109 from the catheter assembly 149 with the other.

As shown in FIG. 20, in this embodiment, the stiffener hub 154 can be permitted to move somewhat proximally relative to the housing 152, but is prevented from fully retracting to the starting position by the stop 392. In this manner, a significant length of the stiffener 106 extends distally beyond a distal tip of the needle 104 to shield the needle tip from inadvertent contact (e.g., preventing inadvertent needle sticking of the user).

As shown in FIG. 21, the catheter assembly 149, a distal end of which may remain in the patient, can include the deployed catheter 102, the catheter hub core 141, a valving member 143, and the catheter connection hub 145.

With reference again to, e.g, FIG. 19, in some embodiments, the system 100 can be resettable. Stated otherwise, the non-return feature or stop 392 may be selectively overridable to permit resetting of the device. In the illustrated embodiment, resetting of the device may be achieved by bending the upper housing element 152t and/or the stiffener hub 154 away from the other (or away from each other) to move the stiffener hub 154 proximally past the stop 392 and back to the initial position of FIG. 2. In some instances, the system 100 may be used in a reset state to advance a catheter into a vessel, even after the bond between the tip of the catheter 102 and the needle 104 has been broken. For example, in some instances, the support provided by the stiffener 106 can be sufficient to assist in urging the catheter tip through the vessel wall.

Illustrative methods of using the system 100 have previously been described. Further details of certain of these or other methods will now be described.

A user of the system 100 may remove the system 100 from packaging, at which point the system 100 can be in the pre-deployment state depicted in FIG. 2. The user may prepare the skin of a patient at which an insertion site will be formed according to standard operating procedures. The user may then advance the distal end of the system 100 (such as depicted in FIG. 2) through the skin of a patient and into a vessel in manners such as previously described. Moreover, as previously described, the stiffener 106 can facilitate and/or reduce or avoid deformation of the distal tip of the catheter 102 during such insertion through the skin, as well as though the vessel wall. Once the distal end of the system 100 has been inserted into the vessel by a sufficient amount, a flash of blood will become visible indicating proper introduction into the vessel has been achieved. At this point, the tip of the needle 104 has entered the vessel, and possibly the distal tip of the catheter 102 and the distal tip of the stiffener 106 may have entered the vessel as well. To the extent the catheter 102 has entered the vessel at this point, the depth to which the catheter 102 has been inserted into the lumen of the vessel may be referred to as the introduction depth, initiation depth, preliminary depth, etc.

After viewing the flash of blood, the user may then deploy the catheter 102 over the needle 104 in any of the manners described above. For example, in some methods, the user may first advance the upper actuator 155 forward, relative to the housing 150 (which may be held substantially stationary, steady, stable, fixed, or immobile relative to the patient and/or relative to the vessel), to deploy the catheter 102 to a capture depth within the vessel, and may thereafter advance the lower actuator 222 forward, relative to the housing 150 (which, again, may be held substantially stationary, steady, stable, fixed, or immobile relative to the patient and/or relative to the vessel), to further deploy the catheter to the final indwelling depth within the vessel, and also to assemble the catheter assembly 149. In other methods, the user only utilizes the lower actuator 222 to fully deploy the catheter to the final indwelling depth, and also to assemble the catheter assembly 149. In either case, the user may be alerted that the indwelling depth has been reached via tactile feedback (e.g., difficulty advancing or inability to advance the lower actuator 222) and/or auditory feedback (e.g., clicking of the catheter assembly 149 into place).

FIG. 19 depicts the stiffener hub 154 in the fully advanced position. In many embodiments, forward movement of the stiffener hub 154 is not delimited by the housing 152 directly, but rather, is delimited by the housing 152 indirectly due to interactions and connections housing 152 and the catheter assembly 149. However, in some instances, the stiffener hub 154 may directly contact the lower housing member 152b in the illustrated operational state in an abutting fashion the delimits forward movement of the stiffener hub 154. In some instances, such contact may only occur in situations at the extreme limits of manufacturing tolerances. In other or further instances, it may be desirable to ensure that at least some space exists between any stopping surfaces of the housing 152 and the stiffener hub 154 when the stiffener hub 154 is fully advanced to ensure sufficient runway exists to permit full assembly of the catheter hub.

As previously noted, the lock 392 can retain the stiffener 106 in an advanced state over the tip of the needle 104 when the stiffener hub 154 has been advanced to the fully advanced state. By being restricted to such a forward position, the stiffener hub 154 can effectively cooperate with the lock 392 to keep the stiffener 106 positioned over or past the distal tip of the needle 104 to thereby shield the needle from inadvertent contact in manners such as previously described. Stated otherwise, in some embodiments (such as certain embodiments discussed hereafter), the stiffener hub 154 can cooperate with the housing 152 to prevent the stiffener 106 from exposing the needle tip after a deployment event. Stated otherwise, the stiffener hub 154 and the attached stiffener 106 can be restrained to a position relative to the housing 154 that maintains the stiffener 106 in a shielding orientation relative to the tip of the needle 104—e.g., in a position in which the stiffener 106 extends distally past the distal tip of the needle 104 by an amount sufficient to inhibit or prevent inadvertent contact with the needle tip.

FIGS. 22A and 22B depict the catheter assembly 149 in a fully assembled state, and further depicts a fluid transfer device 290—specifically, a syringe in the illustrative example—being coupled with the hub 145. In the stage of coupling the fluid transfer device 290 to the catheter assembly 149 depicted in FIG. 22A, a distal tip of the male luer 291 is slightly spaced from the contact surface 224 at the proximal end of the proximal extension 223 of the valving member 143. As the fluid transfer device 290 is thereafter rotated clockwise and thereby advanced distally relative to the hub 145, contact is established with the proximal end of the valving member 143.

With reference to FIG. 22B, the male luer 291 can be further advanced distally as the fluid transfer device 290 is rotated onto the catheter connection hub 145. The proximal end of the valving member 143, including the proximal extension 223, is advanced distally by the advancing male luer 291 of the fluid transfer device 290. This can cause the valve 230 to be advanced distally relative to the valve actuation tip 244 of the valve actuation member 202 and/or can compress the material of the proximal annular extension 223 within the chamber 176 of the catheter connection hub 145. The valve 230 can be stretched and/or otherwise deformed as it advances distally relative to the stationary valve actuation member 202 and/or as the material of the proximal annular extension 223 is deformed, thus causing faces of the slit 232 to separate and transitioning the valve 230 to an open configuration. In the closed configuration, the valve 230 the slit 232 can provide a fluid-tight seal, such as discussed elsewhere herein. Opening of the valve 230 establishes fluid communication between the fluid transfer device 290 and the catheter 102.

Subsequent removal of the fluid transfer device 290, e.g., in a manner opposite from that described with respect to attachment of the fluid transfer device 290 to the catheter connection hub 145, permits the valving member 143 to automatically and resiliently return to a natural state, whereby the valve 230 will reseal to the closed state.

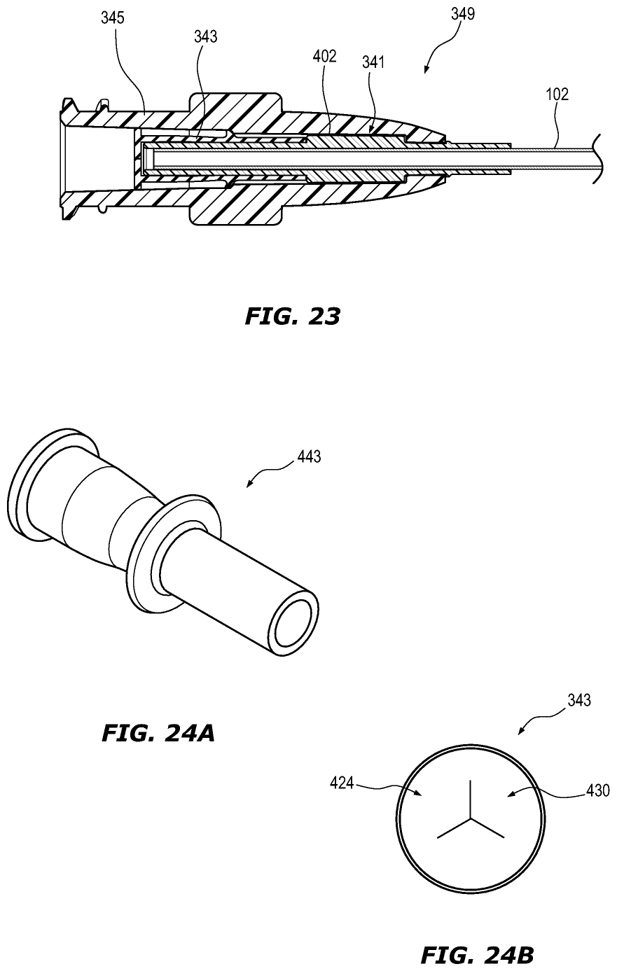

FIG. 23 depicts another embodiment of a catheter assembly 349 that can resemble the catheter assembly 149 in many respects. As discussed below, the catheter assembly 349 includes a valving member 343 and a valve actuation member 402 that vary somewhat from the valving member 143 and the valve actuation member 202.

With reference to FIGS. 23, 24A and 24B, the valving member 343 can include a contact surface 424 that is substantially flush with or coplanar with a proximal face of a valve 430. Stated otherwise a proximal face of the valve 430 can extend outwardly to the peripheral edge of the valving member 343, and a peripheral portion of the valve 430 can define the contact surface 424. The valve 430 can include a tri-slit closable opening. Stated otherwise, the valve 430 can be a tricuspid valve. Other arrangements of the valve are contemplated. For example, in other embodiments, the slit may be a planar slit, such as previously described.

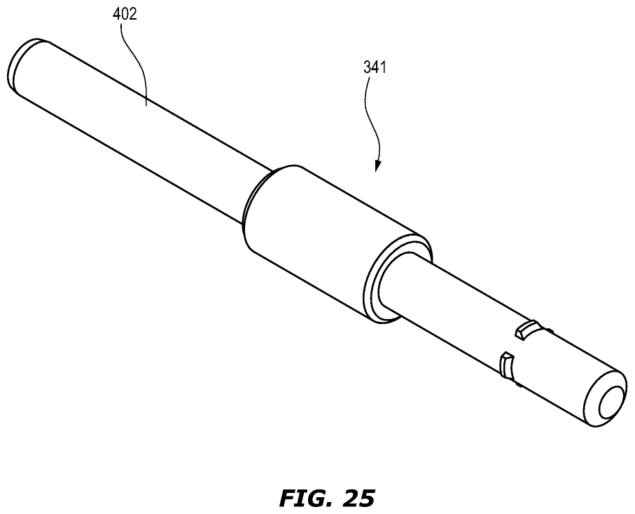

With reference to FIGS. 23 and 25, in the illustrated embodiment, a core 341 is formed as a single unitary member. That is, a separate strain-relief member is not present. Rather, the piercing member 402 extends distally past a distal end of a hub 345 in the fully assembled catheter assembly 349. In some embodiments, the distal end of the piercing member 402 may be more flexible than a proximal end thereof. The proximal end of the piercing member 402 can include a valve actuation tip for opening the valve 430, such as previously described with respect to the piercing member 202.

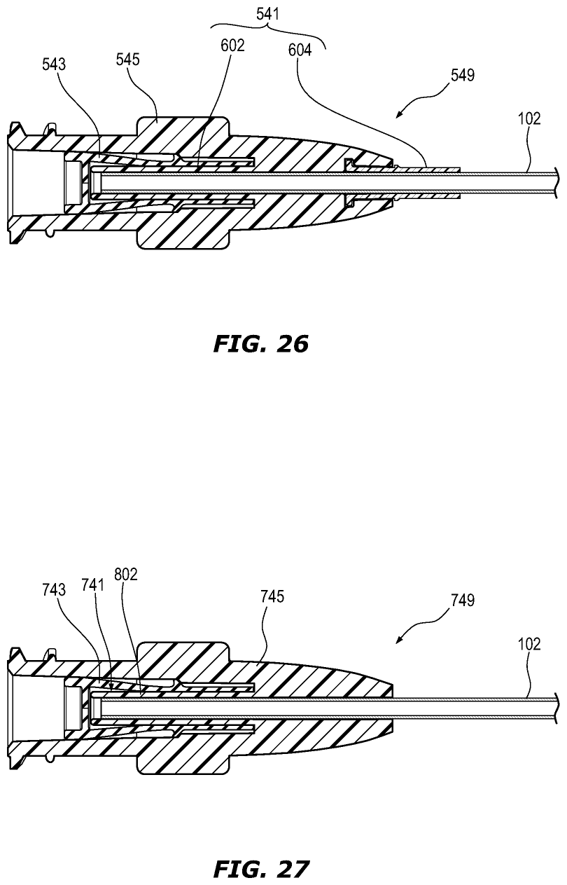

FIG. 26 depicts another embodiment of a catheter assembly 549 that can resemble the catheter assemblies 149, 349 in many respects. In the catheter assembly 549, however, a hub 545 is fixedly secured to the catheter 102 during manufacture. That is, the hub 545 is permanently attached to the catheter 102, or stated otherwise, the hub 545 is attached to the catheter 102 prior to use or placement of the catheter 102. Moreover, a portion of a core 541 is fixedly secured to and integrally formed with the hub 545. The core 541 includes both a piercing member 602, which is a fixedly secured to and integrally formed with the hub 545, and a strain-relief member 604, onto which the hub 545 is overmolded or to which the hub 545 is otherwise attached.

FIG. 27 depicts another embodiment of a catheter assembly 749 that can resemble the catheter assemblies 149, 349, 549 in many respects. In the catheter assembly 749, a hub 745 is fixedly secured to the catheter 102 during manufacture. That is, the hub 745 is permanently attached to the catheter 102, or stated otherwise, the hub 745 is attached to the catheter 102 prior to use or placement of the catheter 102. Moreover, an entirety of a core 741 is fixedly secured to and integrally formed with the hub 745. The core 741 includes a piercing member 802, which is a fixedly secured to and integrally formed with the hub 745. The catheter assembly 749 does not include a strain-relief member.

Various embodiments disclosed herein are suitable for use in power injection procedures. For example, the embodiments can operate at power injection pressures without being damaged, and may be capable of repeated used in such power injection procedures. As used herein, “power injection” is consistent with the generally accepted definition of this term, and refers to pressurized infusions that occur at high flow rates, such as up to 4.0 mL/s or up to 5.0 mL/sec; that often involve injection of viscous materials, such as materials (e.g., contrast media) having a viscosity of 11.8 cP+/−0.3 cP; and that take place at elevated pressures. In like manner, a “power injectable” catheter is one that is capable of sustaining power injection without leaking, bursting, or swelling to a size that is not usable within the vasculature. For example, a power injectable catheter may be one that complies with the power injection specifications of the International Standards Organization (ISO) standard ISO 10555-1.

### EXAMPLES

Following are illustrative examples of devices, systems, and methods consistent with the present disclosure, including the written description and/or drawings.

Example 1. An apparatus comprising:
a hub that defines a cavity;
a catheter that comprises a lumen;
a core attached to the catheter, the core comprising a valve actuation tip, wherein at least a portion of the core is configured to be positioned within the cavity of the hub; and
a valving member comprising an attachment portion that is fixedly attached to the core and a valve positioned distal to a proximal end of the cavity and proximal to the valve actuation tip of the core, the valve being movable relative to the core such that as at least a portion of the valve moves distally, interaction between the valve and the valve actuation tip opens the valve to establish fluid communication between the cavity of the hub and the lumen of the catheter.

Example 2. The apparatus of Example 1, wherein the core is movable relative to the hub from a retracted position to an advanced position.

Example 3. The apparatus of Example 2, wherein the hub is configured to securely couple with the core when the core is moved to the advanced position.

Example 4. The apparatus of Example 2 or Example 3, wherein the core comprises a valve actuation member that defines the valve actuation tip, and wherein an entirety of the valve actuation member is positioned between proximal and distal ends of the hub when the core is in the advanced position.

Example 5. The apparatus of any one of Example 2 to Example 4, wherein the valving member is configured to form a fluid-tight seal with the hub when the core is in at least the advanced position.

Example 6. The apparatus of Example 5, wherein the valving member comprises an annular protrusion that is configured to form the fluid-tight seal with the hub.

Example 7. The apparatus of Example 5 or Example 6, wherein the fluid-tight seal fluidically isolates a proximal portion of the cavity of the hub from a distal portion of the cavity of the hub.

Example 8. The apparatus of any preceding Example, wherein the core comprises a strain-relief member attached to the catheter.

Example 9. The apparatus of Example 8, wherein the strain-relief member comprises a connection region configured to interface with hub to securely couple the core to the hub.

Example 10. The apparatus of any one of Example 2 to Example 9, wherein the core comprises a retention lip.

Example 11. The apparatus of Example 10, wherein the hub comprises a plurality of flexible arms that are configured to:
deflect outwardly to a deflected state as the core is moved distally toward the advanced position and the retention lip passes through the flexible arms; and
return inwardly from the deflected state as the core is moved further distally toward or into the advanced position and the retention lip is moved distally past the flexible arms.

Example 12. The apparatus of Example 10 or Example 11, wherein the retention lip is configured to inhibit proximal movement of the catheter relative to the hub.

Example 13. The apparatus of any one of Example 10 to Example 12, wherein the retention lip comprises a plurality of separate lip elements.

Example 14. The apparatus of any one of Example 2 to Example 13, wherein the core further comprises a stop element.

Example 15. The apparatus of Example 14, wherein the hub comprises one or more stop surfaces that are configured to interface with the stop element to inhibit distal movement of the catheter relative to the hub when the core is in the advanced position.

Example 16. The apparatus of any one of Example 2 to Example 15, further comprising a handle, the hub being removably coupled to the handle.

Example 17. The apparatus of Example 16, further comprising an actuator coupled with the housing and configured to move the core from the retracted position to the advanced position.

Example 18. The apparatus of Example 17, wherein the actuator is configured to advance the catheter into a blood vessel of a patient as the core is moved from the retracted position to the advanced position.

Example 19. The apparatus of Example 17 or Example 18, wherein the actuator is configured to directly contact the valving member to thereby indirectly apply displacement forces to the core and the catheter during at least some portion of a movement event in which the actuator moves the core from the retracted position to the advanced position.

Example 20. The apparatus of Example 19, wherein the actuator comprises a distal tip that is configured to contact a proximal surface of the valve during said at least some portion of the movement event.

Example 21. The apparatus of Example 19 or Example 20, wherein the distal tip of the actuator and the valve actuation tip are configured to contact opposite faces of the valve during said at least some portion of the movement event.

Example 22. The apparatus of any one of Example 16 to Example 21, wherein the hub comprises a connection interface configured for removable coupling with the handle.

Example 23. The apparatus of Example 22, wherein the connection interface comprises threading.

Example 24. The apparatus of Example 22 or Example 23, wherein the connection interface is at a proximal end of the hub.

Example 25. The apparatus of Example 1, wherein the core is fixedly attached to the hub.

Example 26. The apparatus of Example 25, wherein the hub and at least a portion of the core are monolithically formed as a unitary element.

Example 27. The apparatus of Example 26, wherein the hub and the valve actuation member comprise a unitary molded polymeric element.

Example 28. The apparatus of Example 26, wherein the apparatus further comprises a strain-relief member coupled with the hub.

Example 29. The apparatus of any preceding Example, wherein the valving member comprises a protrusion configured to form a fluid-tight seal with the hub when the core and the hub are connected together.

Example 30. The apparatus of Example 29, wherein the protrusion comprises an annular extension that encompasses the core and, when the core and hub are connected together, contacts an inner surface of the of the hub that defines the cavity.

Example 31. The apparatus of Example 29 or Example 30, wherein an inner surface of the hub comprises a luer taper and an inwardly projecting ridge distal to the luer taper, and wherein the protrusion of the valving member contacts the ridge of the hub when the core and the hub are connected together.

Example 32. The apparatus of any preceding Example, wherein the core defines a first lumen and the catheter defines a second lumen in fluid communication with a proximal end of the first lumen.

Example 33. The apparatus of Example 32, wherein a proximal end of the catheter is positioned within the first lumen defined by the core.

Example 34. The apparatus of any preceding Example, wherein a proximal end of the valve actuation tip is positioned proximal to a proximal end of the catheter.

Example 35. The apparatus of any preceding Example, wherein at least the valve actuation tip of the core is rigid.

Example 36. The apparatus of Example 35, wherein the core comprises a valve actuation member that defines the valve actuation tip, and wherein an entirety of the valve actuation member is rigid.

Example 37. The apparatus of any preceding Example, wherein the valve is configured to move distally when a distally directed force is applied thereto, and wherein the valving member further comprises a biasing member configured to automatically move the valve proximally to close the valve after the distally directed force is removed from the valve.

Example 38. The apparatus of Example 37, wherein the biasing member is proximal to the attachment portion of the valving member and distal to the valve of the valving member.

Example 39. The apparatus of Example 37 or Example 38, wherein the biasing member is configured to be compressed in a longitudinal direction, relative to a central axis of the hub, as the valve is moved distally.

Example 40. The apparatus of any one of Example 37 to Example 39, wherein the biasing member comprises a resiliently flexible section of the valving member.

Example 41. The apparatus of any one of Example 37 to Example 40, wherein the biasing member is substantially conically shaped.

Example 42. The apparatus of any one of Example 37 to Example 41, wherein the valving member is formed of a single unitary member that includes each of the attachment portion, the valve, and the biasing member.

Example 43. The apparatus of any one of Example 37 to Example 42, wherein the valving member is formed entirely of silicone.

Example 44. The apparatus of any preceding Example, wherein the valve is movable from an initial proximal position in which the valve is closed to a displaced distal position in which the valve is open.

Example 45. The apparatus of Example 44, wherein the valve is movable from the initial proximal position to the displaced distal position under the influence of a distal displacement force, and wherein the valve is automatically returnable from the displaced distal position to the initial proximal position upon removal of the distal displacement force.

Example 46. The apparatus of Example 44 or Example 45, wherein, when the valve is in the initial proximal position, the valve is in a radially compressed state.

Example 47. The apparatus of Example 46, wherein interaction between the hub and the valving member causes the valve to be in the radially compressed state when the valve is in the initial proximal position.

Example 48. The apparatus of Example 47, wherein the interaction comprises an interference fit between the valving member and the hub.

Example 49. The apparatus of any preceding Example, wherein the valving member is entirely within the hub when the hub and the core are in an assembled state.

Example 50. The apparatus of any preceding Example, wherein the apparatus is configured for power injection through the catheter.

Example 51. The apparatus of Example 50, wherein the valving member is entirely within the hub during power and after said power injection through the catheter.

Example 52. The apparatus of any preceding Example, wherein the attachment portion is at a distal end of the valving member.

Example 53. The apparatus of any preceding Example, wherein the valve is at a proximal end of the valving member.

Example 54. The apparatus of any preceding Example, wherein the valving member further comprises a contact surface at which a male luer can transfer a distal displacement force to the valving member.

Example 55. The apparatus of Example 54, wherein the contact surface is within the hub when the core and the hub are in a coupled state.

Example 56. The apparatus of Example 54 or Example 55, wherein the contact surface is substantially coplanar with a proximal face of the valve.

Example 57. The apparatus of Example 54 or Example 55, wherein a proximal surface of the valve is distally recessed from the contact surface.

Example 58. The apparatus of Example 54, Example 55, or Example 57, wherein the valving member further comprises an annular extension, and wherein the contact surface is at a proximal end of the annular extension.

Example 59. The apparatus of any preceding Example, wherein as the valve actuation tip opens the valve, at least a portion of the valve actuation tip extends through the valve.

Example 60. An apparatus comprising:
a hub that defines a cavity;
a core of which at least a portion is configured to be received within the cavity of the hub, the core comprising a valve actuation tip; and
a valving member comprising:
   an attachment portion that is fixedly attached to the core; and
   a movable portion that comprises a valve positioned longitudinally between a proximal end of the cavity and a proximal end of the valve actuation tip,
wherein the movable portion of the valving member is configured to be directly contacted by a projection of a fluid transfer device when the projection is inserted into the cavity of the hub, such that distal advancement of the projection moves the valve distally to interact with the valve actuation tip so as to be opened thereby.

Example 61. The apparatus of Example 60, wherein the projection of the fluid transfer device comprises a male luer.

Example 62. The apparatus of Example 60 or Example 61, wherein distal movement of the valve causes the valve to impinge on the valve actuation tip to be opened thereby.

Example 63. The apparatus of any one of Example 60 to Example 62, wherein the valving member further comprises a contact surface at which the projection can transfer a distal displacement force to the valving member.

Example 64. The apparatus of Example 63, wherein the contact surface is within the hub when the core and the hub are in a coupled state.

Example 65. The apparatus of Example 63 or Example 64, wherein the contact surface is substantially coplanar with a proximal face of the valve.

Example 66. The apparatus of Example 63 or Example 64, wherein a proximal surface of the valve is distally recessed from the contact surface.

Example 67. The apparatus of Example 63, Example 64, or Example 66, wherein the valving member further comprises an annular extension, and wherein the contact surface is at a proximal end of the annular extension.

Example 68. The apparatus of any one of Example 60 to Example 67, further comprising a catheter attached to the core.

Example 69. An apparatus comprising:
a hub that defines a cavity;
a core of which at least a portion is configured to be received within the cavity of the hub, the core comprising a valve actuation tip;
a catheter coupled to the core; and
a valving member comprising:
   an attachment portion that is fixedly attached to the core;
   a contact extension at a proximal end of the valving member; and
   a valve distally recessed from a proximal end of the contact extension, the valve being movable relative to the core when distal force is applied to the contact extension such that as the valve moves distally relative to the valve actuation tip, the valve actuation tip opens the valve.

Example 70. The apparatus of Example 69, wherein the contact extension is shaped as a hollow cylinder.

Example 71. The apparatus of Example 69 or Example 70, wherein the catheter defines a first lumen, the contact extension defines a second lumen, and the valve fluidically isolates the first lumen from the second lumen when closed.

Example 72. The apparatus of Example 71, wherein the valve permits fluid communication between the first lumen and the second lumen when open.

Example 73. An apparatus comprising:
a hub that defines a cavity;
a core of which at least a portion is configured to be received within the cavity of the hub, the core comprising a valve actuation tip; and
a valving member comprising:
   an attachment portion that is fixedly attached to the core; and
   a resilient valve positioned within the cavity of the housing so as to be radially compressed by the housing and maintained in a closed state thereby when in a proximal position, the valve being movable relative to the core when distal force is applied to the valving member such that as the valve moves distally relative to the valve actuation tip, the valve actuation tip opens the valve.

Example 74. An apparatus comprising:
a hub comprising a connector; and
a catheter assembly moveable relative to the hub from a retracted position to an advanced position, the catheter assembly comprising:
   a catheter that extends through the hub when the catheter assembly is in the retracted position;
   a core attached to a proximal end of the catheter;
   a valving member fixedly secured to the core, the valving member comprising a valve configured to be openable by the core when moved distally relative to the core; and
   a connection region,
wherein movement of the catheter assembly from the retracted position to the advanced position advances the connection region of the catheter assembly distally into connective engagement with the connector of the hub to fixedly secure the catheter assembly to the hub.

Example 75. The apparatus of Example 74, further comprising a handle, wherein the hub is removably coupled to the handle.

Example 76. The apparatus of Example 75, wherein the hub is configured to be decoupled from the handle after the catheter assembly has been moved to the advanced position and fixedly secured to the hub.

Example 77. The apparatus of any one of Example 74 to Example 76, further comprising an actuator that is movable relative to the handle, wherein the actuator is coupled with the catheter assembly to move the actuator assembly from the retracted position to the advanced position.

Example 78. The apparatus of Example any one of Example 74 to Example 77, wherein the connector comprises a plurality of resilient arms.

Example 79. The apparatus of Example 78, wherein the connection region comprises at least one lateral protrusion that urges the plurality of resilient arms laterally outwardly as the catheter assembly is moved to the advanced position.

Example 80. A method of assembling a catheter assembly, the method comprising:
advancing a catheter through a hub;
subsequently advancing a portion of a core that is attached to the catheter through the hub, wherein the core comprises a connection region and is attached to a valving member that is configured to provide selective fluid communication to a lumen of the catheter; and
automatically connecting the hub to the connection region of the core.

Example 81. The method of Example 80, wherein the hub comprises a plurality of resilient arms, and wherein said automatically connecting the hub to the connection region of the core comprises permitting the resilient arms to deflect radially inwardly after having been deflected outwardly due to said subsequently advancing a portion of the core that is attached to the catheter through the hub.

Example 82. The method of Example 80 or Example 81, wherein said advancing the catheter through the hub comprises advancing an actuator that is in direct contact with the valving member.

Example 83. The method of Example 82, wherein the actuator is in direct contact with a valve portion of the valving member during said advancing the catheter through the hub.

It will be understood by those having skill in the art that changes may be made to the details of the above-described embodiments without departing from the underlying principles presented herein. For example, any suitable combination of various embodiments, or the features thereof, is contemplated.

Although the foregoing detailed description contains many specifics for the purpose of illustration, a person of ordinary skill in the art will appreciate that many variations and alterations to the following details can be made and are considered to be included herein. Accordingly, the foregoing embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

As used in this specification and the appended claims, the singular forms “a,” “an” and “the” include plural referents unless the context clearly dictates otherwise. Thus, for example, reference to “a layer” can include a plurality of such layers.

In this disclosure, “comprises,” “comprising,” “containing” and “having” and the like can have the meaning ascribed to them in U.S. patent law and can mean “includes,” “including,” and the like, and are generally interpreted to be open ended terms. The terms “consisting of” or “consists of” are closed terms, and include only the component structures, steps, or the like specifically listed in conjunction with such terms, as well as that which is in accordance with U.S. patent law.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments described herein are, for example, capable of operation in other orientations than those illustrated or otherwise described herein. The term “coupled,” as used herein, is defined as directly or indirectly connected in any suitable manner. Objects described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

As used herein, the term “substantially” refers to the complete or nearly-complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Moreover, for references to approximations (which are made throughout this specification), such as by use of the terms “about” or “approximately,” or other terms, it is to be understood that, in some embodiments, the value, feature, or characteristic may be specified without approximation. For example, where qualifiers such as “about,” “substantially,” and “generally” are used, these terms include within their scope the qualified words in the absence of their qualifiers. For example, where the term “substantially perpendicular” is recited with respect to a feature, it is understood that in further embodiments, the feature can have a precisely perpendicular orientation.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

References throughout this specification to “an example,” if any, mean that a particular feature, structure, or characteristic described in connection with the example is included in at least one embodiment. Thus, appearances of the phrases “in an example” in various places throughout this specification are not necessarily all referring to the same embodiment.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description. These additional embodiments are determined by replacing the dependency of a given dependent claim with the phrase “any of claims [x] through the claim that immediately precedes this one” where the bracketed term “[x]” is replaced with the number of the most recently recited independent claim. For example, for the first claim set that begins with independent claim 1, claim 3 can depend from either of claims 1 and 2, with these separate dependencies yielding two distinct embodiments; claim 4 can depend from any one of claim 1, 2, or 3, with these separate dependencies yielding three distinct embodiments; claim 5 can depend from any one of claim 1, 2, 3, or 4, with these separate dependencies yielding four distinct embodiments; and so on.

Recitation in the claims of the term “first” with respect to a feature or element does not necessarily imply the existence of a second or additional such feature or element. Elements specifically recited in means-plus-function format, if any, are intended to be construed in accordance with 35 U.S.C. § 112(f). Elements not presented in requisite means-plus-function format are not intended to be construed in accordance with 35 U.S.C. § 112(f). Embodiments of the invention in which an exclusive property or privilege is claimed are defined as follows.

1. An apparatus comprising:
a hub that defines a cavity;
a catheter that comprises a lumen;
a core attached to the catheter, the core comprising a valve actuation tip, wherein at least a portion of the core is configured to be positioned within the cavity of the hub; and
a valving member comprising an attachment portion that is fixedly attached to the core and a valve positioned distal to a proximal end of the cavity and proximal to the valve actuation tip of the core, the valve being movable relative to the core such that as at least a portion of the valve moves distally, interaction between the valve and the valve actuation tip opens the valve to establish fluid communication between the cavity of the hub and the lumen of the catheter.

2. The apparatus of claim 1, wherein the core is movable relative to the hub from a retracted position to an advanced position.

3. The apparatus of claim 2, wherein the hub is configured to securely couple with the core when the core is moved to the advanced position.
